SEQUENCE LISTING (59 sequences)

SEQ ID NO: 1 - DNA, 3804 bp, Canis familiaris
agctaccaagacaagaaaaaaatgaagagaacgaagctgattggatcattaactttgtttagatattctgactggcaggataaattattgatgtccctgggcactatcatggccatagctcatggatcaggtctccccctcatgatgatcgtttttggacaaatgactgacaaatttgtggatactgcaggaaacttctcctttccagtgaatttttcattatcaatgctaaatccaggcagaattctggaagaagaaatgactaggtatgcatattattactcagggttaggtgctggagttctcgttgctgcctatatacaagtttcattttggactctggcagccggccgacagatcaggaaaattagacaggagttttttcatgctattctacgacaagaaataggctggtttgatgtcaatgacaccactgaacttaatacacggttaacagatgacatctccaaaatcagtgaaggaattggtgacaaggttggaatgttctttcaagcagtagccacgttttttgcaggattcatagtagggttcgttagaggatggaagctcacccttgtgataatggccatcagccctattctgggactctctgcagctgtttgggcaaagatactctcggcatttagtgacaaagaactagctgcatatgccaaagcaggagctgtggcggaagaggctctgggggccatcaggaccgtgatagcctttggaggccagaacaaagagctgaagaggtatgagaaatatctagaacatgccaaaaagatgggaattaaaaaagctatttccgcaaacatctccatgggcatcgccttcctgttaatatatgcatcatatgcgctggccttctggtatggatccactttagttatatcaaaagaatatactattggaaatgcaatgacagtctttttttcaatcctgattggagccttcagtgtgggacaggctgctccatgtattgactcttttgccaatgcaagaggagccgcctatgcgatcttcaacattattgacagtaatcctaaaattgacagtttttcagagagaggacacaaacctgacagcatcaaagggaatttggagttcattgatgttcatttttcttatccagctcgagctgatgtaaagatcttgaagggtctcaacctgaaggttcagagcgggcagacggtggcgctggttgggaacagtggctgtgggaaaagtacaaccgtgcagctgatgcagaggctctatgaccctgatgagggtatgattaacattgatgggcaggatattaagacctttaatgtaaggtatctcagggaaataattggagtggtgagtcaggagccagtgctgttttctaccacaattgctgaaaatattcgctatggccgtggaaatgtcaccatggatgagataaagaaagctgtcaaagaagccaatgcctatgagtttatcatgaaactaccacagaaatttgacaccctggttggagagagaggggcccagctgagtggtggacagaaacagagaattgccattgcgagggccctggttcgcaaccctaagatcctcctgctggatgaagccacgtcagccctggacactgaaagtgaagcagaggtacaggcggccctggataaggccagagaaggccggaccaccatcgtaatagcccatcgactgtccactatccgaaatgcagatgtcattgctgggtttgaggatggcgtcattgtggagcaaggaaaccacagggagctgatgaagaaggagggggtgtacttcaagcttgttaacatgcagacctcaggaaaccaaacccaatcaggagaatttgatgttgaactaaataatgaaaaggctgttggggataaggctccaaatggctggaaatctcgcatatttaggaattctacacagaaaagccttaggaattcacgaaagtatcataatggccttgatgtagaaagcaaagaacttgatgaaaatgtgccatcagtgtcttttctgaaggtcttgaaattgaataaaacagaatggccgtactttgtgataggcaccatgtgcgccattgccaacggggcgcttcagccagcgttttccatcatattctcagaaatgatagcggtttttggaccaggcgatgatgaagtgaagcagcagaaatgcaacatgttttcattgctttttttaggtctgggaattatttccttttttactttcttccttcagggcttcacttttgggaaagctggcgagatcctcaccacaaggcttcgatcattggctttcagagcaatgctaagacaggacatgagctggtttgatgatcataaaaacagtactggtgcgctttctacaagacttgcaactgatgcctcccaggtccaaggagccactgggatgaggttggctttaattgcccagaacacagctaacctcggaactggtattatcatatcatttatctacggttggcagctgacccttctgctgttagtggttgtgccagtcattgctgtatcaggaattgttgaaatgaaaatgttggctggaaatgccaagagagataaaaaagaactggaaactgctggaaagattgcaacagaagcaatagaaaatattaggacagttgtgtccttagcccgggaaagaaaatttgaatcgatgtatgttgaaaagttatacggagcttacaggaattctgtgcggaaggcacacatctatggaattacttttagtatttcacaagcatttatgtatttttcctatgccggttgttttcgatttggtgcctatctcattgtgaatggacatatgcgctttagagaggttattctggtgttttcagcaatcgtgtttggtgcagtggctctcggacatgccagttcctttgcgccagactatgctaaagccaagctgtctgccgcccacttgttcatgctgcttgaaagacaacctctgattgacagctacagtgaagaaggcctgaggcctgataaatttgaaggaaatgtgacatttaatgaagtcatgttcaactatcccacgcggccaaaagtgcccgtgctccaggggctgagtcttaaggtgaagaagggccagacgctggccctggtcggcagcagtggctgtgggaagagcacagtggtccagctcctagagcgcttctatgacccagtcgctggaacagtgtttgtggactttggttttcagctcttagatggtcaagaagcaaagaaacttaacatccagtggctcagagcccaccttggaattgtgtcccaggagcctgtcctgtttgactgcagcattgctgagaatattgcctatggagacaatagtcgggctgtctcacaggatgaaattgtgaatgcagccaaagcagccaacatacatcctttcatcgagaccttaccccataaatatgaaacaagagtgggagataagggaactcagctctcaggaggtcaaaacaaaagatgctatcgtcgagccctcatcagacagctcaaaatcctatgtaaggatgaagctacatcagctctggatactgaaagtgaaaagattgtccaagaagccctggacaaagcacgagaaggccggacctgtatcgtgattgcccaccgcctgtccaccatccagaatgcagacataatagtggtgtttcagaatggcaaagtcaaggagcatggcacgcatcaacagctgctggctcagaaaggcatctatttttccatggtcagtgtccagactggaacacagaacttaggaactcttgctatatcttaa

SEQ ID NO: 2 - protein, 1260 aa, Canis familiaris
MKRTKLIGSLTLFRYSDWQDKLLMSLGTIMAIAHGSGLPLMMIVFGQMTDKFVDTAGNFSFPVNFSLSMLNPGRILEEEMTRYAYYYSGLGAGVLVAAYIQVSFWTLAAGRQIRKIRQEFFHAILRQEIGWFDVNDTTELNTRLTDDISKISEGIGDKVGMFFQAVATFFAGFIVGFVRGWKLTLVIMAISPILGLSAAVWAKILSAFSDKELAAYAKAGAVAEEALGAIRTVIAFGGQNKELKRYEKYLEHAKKMGIKKAISANISMGIAFLLIYASYALAFWYGSTLVISKEYTIGNAMTVFFSILIGAFSVGQAAPCIDSFANARGAAYAIFNIIDSNPKIDSFSERGHKPDSIKGNLEFIDVHFSYPARADVKILKGLNLKVQSGQTVALVGNSGCGKSTTVQLMQRLYDPDEGMINIDGQDIKTFNVRYLREIIGVVSQEPVLFSTTIAENIRYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTIRNADVIAGFEDGVIVEQGNHRELMKKEGVYFKLVNMQTSGNQTQSGEFDVELNNEKAVGDKAPNGWKSRIFRNSTQKSLRNSRKYHNGLDVESKELDENVPSVSFLKVLKLNKTEWPYFVIGTMCAIANGALQPAFSIIFSEMIAVFGPGDDEVKQQKCNMFSLLFLGLGIISFFTFFLQGFTFGKAGEILTTRLRSLAFRAMLRQDMSWFDDHKNSTGALSTRLATDASQVQGATGMRLALIAQNTANLGTGIIISFIYGWQLTLLLLVVVPVIAVSGIVEMKMLAGNAKRDKKELETAGKIATEAIENIRTVVSLARERKFESMYVEKLYGAYRNSVRKAHIYGITFSISQAFMYFSYAGCFRFGAYLIVNGHMRFREVILVFSAIVFGAVALGHASSFAPDYAKAKLSAAHLFMLLERQPLIDSYSEEGLRPDKFEGNVTFNEVMFNYPTRPKVPVLQGLSLKVKKGQTLALVGSSGCGKSTVVQLLERFYDPVAGTVFVDFGFQLLDGQEAKKLNIQWLRAHLGIVSQEPVLFDCSIAENIAYGDNSRAVSQDEIVNAAKAANIHPFIETLPHKYETRVGDKGTQLSGGQNKRCYRRALIRQLKILCKDEATSALDTESEKIVQEALDKAREGRTCIVIAHRLSTIQNADIIVVFQNGKVKEHGTHQQLLAQKGIYFSMVSVQTGTQNLGTLAIS

SEQ ID NO: 3 - DNA, 1654 bp, Canis familiaris
agctaccaagacaagaaaaaaatgaagagaacgaagctgattggatcattaactttgtttagatattctgactggcaggataaattattgatgtccctgggcactatcatggccatagctcatggatcaggtctccccctcatgatgatcgtttttggacaaatgactgacaaatttgtggatactgcaggaaacttctcctttccagtgaatttttcattatcaatgctaaatccaggcagaattctggaagaagaaatgactaggtatgcatattattactcagggttaggtgctggagttctcgttgctgcctatatacaagtttcattttggactctggcagccggccgacagatcaggaaaattagacaggagttttttcatgctattctacgacaagaaataggctggtttgatgtcaatgacaccactgaacttaatacacggttaacagatgacatctccaaaatcagtgaaggaattggtgacaaggttggaatgttctttcaagcagtagccacgttttttgcaggattcatagtagggttcgttagaggatggaagctcacccttgtgataatggccatcagccctattctgggactctctgcagctgtttgggcaaagatactctcggcatttagtgacaaagaactagctgcatatgccaaagcaggagctgtggcggaagaggctctgggggccatcaggaccgtgatagcctttggaggccagaacaaagagctgaagaggtatgagaaatatctagaacatgccaaaaagatgggaattaaaaaagctatttccgcaaacatctccatgggcatcgccttcctgttaatatatgcatcatatgcgctggccttctggtatggatccactttagttatatcaaaagaatatactattggaaatgcaatgacagtctttttttcaatcctgattggagccttcagtgtgggacaggctgctccatgtattgactcttttgccaatgcaagaggagccgcctatgcgatcttcaacattattgacagtaatcctaaaattgacagtttttcagagagaggacacaaacctgacagcatcaaagggaatttggagttcattgatgttcatttttcttatccagctcgagctgatgtaaagatcttgaagggtctcaacctgaaggttcagagcgggcagacggtggcgctggttgggaacagtggctgtgggaaaagtacaaccgtgcagctgatgcagaggctctatgaccctgatgagggtatgattaacattgatgggcaggatattaagacctttaatgtaaggtatctcagggaaataattggagtggtgagtcaggagccagtgctgttttctaccacaattgctgaaaatattcgctatggccgtggaaatgtcaccatggatgagataaagaaagctgtcaaagaagccaatgcctatgagtttatcatgaaactaccacagaaatttgacaccctggttggagagagaggggcccagctgagtggtggacagaaacagagaattgccattgcgagggccctggttcgcaaccctaagatcctgcctgctggatgaagccacgtcagccctggacactgaaagtgaagcagaggtacaggcggccctggataag

SEQ ID NO: 4 - protein, 524 aa, Canis familiaris
MKRTKLIGSLTLFRYSDWQDKLLMSLGTIMAIAHGSGLPLMMIVFGQMTDKFVDTAGNFSFPVNFSLSMLNPGRILEEEMTRYAYYYSGLGAGVLVAAYIQVSFWTLAAGRQIRKIRQEFFHAILRQEIGWFDVNDTTELNTRLTDDISKISEGIGDKVGMFFQAVATFFAGFIVGFVRGWKLTLVIMAISPILGLSAAVWAKILSAFSDKELAAYAKAGAVAEEALGAIRTVIAFGGQNKELKRYEKYLEHAKKMGIKKAISANISMGIAFLLIYASYALAFWYGSTLVISKEYTIGNAMTVFFSILIGAFSVGQAAPCIDSFANARGAAYAIFNIIDSNPKIDSFSERGHKPDSIKGNLEFIDVHFSYPARADVKILKGLNLKVQSGQTVALVGNSGCGKSTTVQLMQRLYDPDEGMINIDGQDIKTFNVRYLREIIGVVSQEPVLFSTTIAENIRYGRGNVTMDEIKKAVKEANAYEFIMKLPQKFDTLVGERGAQLSGGQKQRIAIARALVRNPKILPAG

SEQ ID NO: 5 - DNA, 24 bp, Artificial sequence
  Synthetic oligonucleotide
cctggttcgcaaccctaagatccg

SEQ ID NO: 6 - DNA, 29 bp, Artificial sequence
  Synthetic oligonucleotide
gcaatgtggcctgacagaaaggggaaatc

SEQ ID NO: 7 - DNA, 23 bp, Artificial sequence
  Synthetic oligonucleotide
cctggttcgcaaccctaagatcc

SEQ ID NO: 8 - DNA, 24 bp, Artificial sequence
  Synthetic oligonucleotide
ttcagttggctatgaaacatttgg

SEQ ID NO: 9 - DNA, 24 bp, Artificial sequence
  Synthetic oligonucleotide
agactatcttaaagcactgactcc

SEQ ID NO: 10 - DNA, 24 bp, Artificial sequence
  Synthetic oligonucleotide
ccaaaaaacatatagttttgggga

SEQ ID NO: 11 - DNA, 27 bp, Artificial sequence
  Synthetic oligonucleotide
gtcatctagaagtgcaaaccattaaac

SEQ ID NO: 12 - DNA, 28 bp, Artificial sequence
  Synthetic oligonucleotide
cctagtaacacctattaatagttcagcc

SEQ ID NO: 13 - DNA, 24 bp, Artificial sequence
  Synthetic oligonucleotide
ctctgtaagtttgcaattattctc

SEQ ID NO: 14 - DNA, 26 bp, Artificial sequence
  Synthetic oligonucleotide
cttcctgaaagagatgaataaagaac

SEQ ID NO: 15 - DNA, 29 bp, Artificial sequence
  Synthetic oligonucleotide
caaaagtatgacataaatgatacacttac

SEQ ID NO: 16 - DNA, 24 bp, Artificial sequence
  Synthetic oligonucleotide
gaagacctcctgcctgtaaccact

SEQ ID NO: 17 - DNA, 24 bp, Artificial sequence
  Synthetic oligonucleotide
cacatgtgaaaatgttcccgtttc

SEQ ID NO: 18 - DNA, 24 bp, Artificial sequence
  Synthetic oligonucleotide
catgaatgtttcttctctgtccag

SEQ ID NO: 19 - DNA, 20 bp, Artificial sequence
  Synthetic oligonucleotide
ggttctttgaaccagtggac

SEQ ID NO: 20 - DNA, 26 bp, Artificial sequence
  Synthetic oligonucleotide
ggctatgattatggactgttttcttg

SEQ ID NO: 21 - DNA, 26 bp, Artificial sequence
  Synthetic oligonucleotide
ggtttcttcacgaatattagaaagac

SEQ ID NO: 22 - DNA, 28 bp, Artificial sequence
  Synthetic oligonucleotide
gcttataacttcttcttgtgttcttttg

SEQ ID NO: 23 - DNA, 26 bp, Artificial sequence
  Synthetic oligonucleotide
gtgcaagcctcaaggaattttttttg

SEQ ID NO: 24 - DNA, 21 bp, Artificial sequence
  Synthetic oligonucleotide
ccttaaaagtgcagttggttg

SEQ ID NO: 25 - DNA, 20 bp, Artificial sequence
  Synthetic oligonucleotide
gaaataaaacctgccacagg

SEQ ID NO: 26 - DNA, 21 bp, Artificial sequence
  Synthetic oligonucleotide
cgtgaagagtgttctctttct

SEQ ID NO: 27 - DNA, 18 bp, Artificial sequence
  Synthetic oligonucleotide
gcagggctaattggtagc

SEQ ID NO: 28 - DNA, 24 bp, Artificial sequence
  Synthetic oligonucleotide
cttgatgctttagatgtcagatgg

SEQ ID NO: 29 - DNA, 21 bp, Artificial sequence
  Synthetic oligonucleotide
ctcacttgcctgaagtcaaag

SEQ ID NO: 30 - DNA, 22 bp, Artificial sequence
  Synthetic oligonucleotide
gagatacatcaggagctcctcc

SEQ ID NO: 31 - DNA, 20 bp, Artificial sequence
  Synthetic oligonucleotide
caggtgtttcgggttgactg

SEQ ID NO: 32 - DNA, 20 bp, Artificial sequence
  Synthetic oligonucleotide
gtaaccctgttgcatcacac

SEQ ID NO: 33 - DNA, 20 bp, Artificial sequence
  Synthetic oligonucleotide
ctcagcatggcattagctgc

SEQ ID NO: 34 - DNA, 26 bp, Artificial sequence
  Synthetic oligonucleotide
caacttaacattttctcttctttcag

SEQ ID NO: 35 - DNA, 18 bp, Artificial sequence
  Synthetic oligonucleotide
ggaatcacttgtgcctgc

SEQ ID NO: 36 - DNA, 22 bp, Artificial sequence
  Synthetic oligonucleotide
ccactttctcctgattctcctg

SEQ ID NO: 37 - DNA, 20 bp, Artificial sequence
  Synthetic oligonucleotide
ggtgaagctggcatgagaac

SEQ ID NO: 38 - DNA, 18 bp, Artificial sequence
  Synthetic oligonucleotide
ctctctctggctctcatg

SEQ ID NO: 39 - DNA, 23 bp, Artificial sequence
  Synthetic oligonucleotide
ctctaatagaatgtggactcgag

SEQ ID NO: 40 - DNA, 22 bp, Artificial sequence
  Synthetic oligonucleotide
ctgatgatcaaaagggacaatc

SEQ ID NO: 41 - DNA, 19 bp, Artificial sequence
  Synthetic oligonucleotide
ggacttctcaagtgcacac

SEQ ID NO: 42 - DNA, 22 bp, Artificial sequence
  Synthetic oligonucleotide
gaaggtgtgttttgtgccacag

SEQ ID NO: 43 - DNA, 22 bp, Artificial sequence
  Synthetic oligonucleotide
ccctttctgtctctcaaatggg

SEQ ID NO: 44 - DNA, 23 bp, Artificial sequence
  Synthetic oligonucleotide
catggctccctctttgcttttgc

SEQ ID NO: 45 - DNA, 25 bp, Artificial sequence
  Synthetic oligonucleotide
ctcactgaagccttctttgacccac

SEQ ID NO: 46 - DNA, 22 bp, Artificial sequence
  Synthetic oligonucleotide
cgttatccagaagtaaaagccc

SEQ ID NO: 47 - DNA, 21 bp, Artificial sequence
  Synthetic oligonucleotide
cctcaggaaagtactagggtc

SEQ ID NO: 48 - DNA, 23 bp, Artificial sequence
  Synthetic oligonucleotide
ccagtcaactacactagaagctg

SEQ ID NO: 49 - DNA, 23 bp, Artificial sequence
  Synthetic oligonucleotide
gaacaagtgagttttttccaccc

SEQ ID NO: 50 - DNA, 22 bp, Artificial sequence
  Synthetic oligonucleotide
ggtaagcactatgtctttggac

SEQ ID NO: 51 - DNA, 22 bp, Artificial sequence
  Synthetic oligonucleotide
cattcaccagacagcagagaac

SEQ ID NO: 52 - DNA, 27 bp, Artificial sequence
  Synthetic oligonucleotide
cagaccaattataatagcaacattaac

SEQ ID NO: 53 - DNA, 26 bp, Artificial sequence
  Synthetic oligonucleotide
gccttaaataaggtactaacttaagc

SEQ ID NO: 54 - DNA, 24 bp, Artificial sequence
  Synthetic oligonucleotide
gatacccacatgtcacaatgttcc

SEQ ID NO: 55 - DNA, 21 bp, Artificial sequence
  Synthetic oligonucleotide
tcctggtgccactacatagac

SEQ ID NO: 56 - DNA, 23 bp, Artificial sequence
  Synthetic oligonucleotide
gtcctataccaagtcatgaggac

SEQ ID NO: 57 - DNA, 20 bp, Artificial sequence
  Synthetic oligonucleotide
ggaaacagagtggaaagacc

SEQ ID NO: 58 - DNA, 24 bp, Artificial sequence
  Synthetic oligonucleotide
ggaactaactgtagactataatgc

SEQ ID NO: 59 - DNA, 23 bp, Artificial sequence
  Synthetic oligonucleotide
gctatcttatcaacaccaaatgg

(19) United States
(12) Patent Application Publication
(10) Pub. No.: US 2011/0038944 A1
(43) Pub. Date: Feb. 17, 2011

(54) COMPOSITIONS AND METHODS FOR DIAGNOSIS AND TREATMENT OF HEPATOBILIARY DISEASE AND ASSOCIATED DISORDERS

(75) Inventors: KATRINA L. MEALEY, PULLMAN, WA (US); JONATHAN MINCH, PULLMAN, WA (US)

Correspondence Address: KLARQUIST SPARKMAN, LLP, 121 SW SALMON STREET, SUITE 1600, PORTLAND, OR, 97204, US

(73) Assignee: WASHINGTON STATE UNIVERSITY RESEARCH FOUNDATION

(21) Appl. No.: 12/856,095

(22) Filed: Aug. 13, 2010

Related U.S. Application Data
(60) Provisional application No. 61/233,752, filed on Aug. 13, 2009.

(51) Int. Cl. A61K 35/413; A61P 1/16; C12Q 1/68

(52) U.S. Cl. 424/528; 435/6

(57) ABSTRACT

Provided are nucleic acids comprising ABCB4 gene mutations (e.g., insertions, deletions, frame shift, missense) (e.g., canine ABCB4 1583_1584G) that result in prematurely terminated and/or inactive ABCB4 proteins. A significant association (P=1.54E-7) of the mutations was detected between gallbladder mucoceles-affected dogs, providing methods for detection or diagnosis of a hepatobiliary disease and/or related conditions (e.g., increased mucin secretion, mucinous hyperplasia, gallbladder mucocele, progressive familial intrahepatic cholestasis (type 3), cholelithiasis, primary biliary cirrhosis, and intrahepatic cholestasis of pregnancy). Compositions and methods for genotyping or screening of test subjects are provided, and have added utility in combination with surgical intervention, selective breeding, and medical or dietary management. Also provided are methods of treatment of dogs with hepatobiliary disease, comprising administration of hydrophilic, less cytotoxic bile acids (e.g., ursodeoxycholate). Methods for screening or testing for therapeutic agents for treatment of human hepatobiliary disease, comprising use of canine test subjects harboring ABCB4 gene mutations are provided.

### CROSS REFERENCE TO RELATED APPLICATION

This application claims the benefit of U.S. Provisional Application No. 61/223,752 filed Aug. 13, 2009, which is incorporated by reference herein in its entirety.

### FIELD

This disclosure relates to methods for diagnosis and treatment of hepatobiliary disease and associated disorders, for instance detection of novel mutations (e.g., an insertion mutation) in the canine ABCB4 gene that are associated with gallbladder disease in dogs. Further, it relates to methods of breeding dogs to decrease the prevalence of gallbladder disease in a dog population.

### BACKGROUND

Bile is produced by the collective actions of a number of transporters located on canalicular membrane of hepatocytes (Pellicoro et al., Aliment Pharmacol Ther., 26 Suppl 2: 149-60, 2007). Active transport of biliary solutes creates an osmotic force that attracts water through tight junctions and aquaporins in the hepatocyte membrane (Coleman et al., Biochem J., 178: 201-208, 1979 and Elferink et al., Biochim. Biophys. Acta. 1586: 129-145, 2002). Bile salts are the most important biliary solute. Other important solutes of bile include cholesterol, and phospholipids. The presence of phospholipids, phosphatidylcholine (PC) in particular, in the biliary lumen is crucial for protecting the epithelial cell membranes lining the biliary system from the cytotoxic detergent actions of bile salts (Coleman et al., Biochem J., 178: 201-208, 1979, Davit-Spraul et al., Orphanet. J. Rare Dis., 4: 1, 2009, and Oude Elferink et al., Pflugers. Arch., 453: 601-610, 2007). Bile salt cytotoxicity is substantially reduced in the presence of PC owing to the formation of mixed micelles (PC+bile salts) rather than simple micelles (bile salts only). Thus, a decrease in the amount of biliary PC leads to injury of epithelial cells lining the biliary system (Baghdasaryan et al., Liver Int., 28: 948-958, 2008).

Biliary disease, specifically gallbladder mucocele formation, has been recognized with increased frequency during the past decade, and particularly in dogs. For example, Shetland Sheepdogs are considered to be predisposed to gallbladder mucoceles. Several diseases of the gall bladder and biliary system in people are known to be caused by defects in the ABCB4 gene.

The ABCB4 gene product is a lipid translocator that is essential for transporting phospholipids into the bile. Phospholipids help protect the biliary system by buffering both cholesterol and bile salts. Lack of phospholipids in bile can result in gallbladder stones, cirrhosis, and jaundice. Patients may be mildly or severely affected, requiring liver transplantation. The only known physiologic function of the ABCB4 gene product is translocation of phosphatidylcholine (PC) across the hepatocyte plasma membrane into biliary canaliculi (Trauner et al., Semin. Liver Dis., 27: 77-98, 2007). ABCB4 is expressed on canalicular membranes of hepatocytes where it translocates PC from the hepatocyte to the biliary canalicular lumen (Dean et al., Ann. Rev. Genomics Hum. Genet., 6: 123-142, 2005). Proper function of ABCB4 is critical for maintaining hepatobiliary homeostasis as evidenced by the myriad of diseases that occur when polymorphisms of ABCB4 cause complete or partial protein dysfunction.

In people, ABCB4 deficiency or mutations of the ABCB4 gene produce several disease syndromes involving the biliary system including progressive familial intrahepatic cholestasis (type 3), cholelithiasis, primary biliary cirrhosis, and intrahepatic cholestasis of pregnancy (Delaunay et al., Hepatology, 49: 1218-1227, 2009, Gonzales et al, Front Biosci., 14: 4242-4256, 2009, Nakken et al., Liver Int., 29: 743-747, 2009, and Oude Elferink et al., Pflugers. Arch., 453: 601-610, 2007). The severity of the disease can range from fatal (without a liver transplant) to milder forms depending on whether the mutation reduces or completely eliminates ABCB4 protein function. In severely affected people, the disease manifests during early childhood and is fatal without a liver transplant (Oude Elferink et al., Pflugers. Arch., 453: 601-610, 2007). Abcb4−/− mice also develop hepatobiliary disease (Baghdasaryan et al., Liver Int, 28: 948-958, 2008 and Popov et al., J. Hepatol., 43: 1045-1054, 2005).

Hepatobiliary disease in dogs has been recognized with increased frequency during the past several years. In particular, gall bladder mucoceles (mucinous hyperplasia or mucinous cholecystitis) have been documented to be an increasingly important cause of hepatobiliary disease in dogs (Aguirre et al., J. Am. Vet. Med. Assoc., 231: 79-88, 2007, Besso et al., Vet. Radiol. Ultrasound, 41: 261-271, 2000, and Pike et al., J. Am. Vet. Med. Assoc., 224: 1615-1622, 2004). The etiology of gallbladder mucoceles, which are uncommon in people, has not yet been identified. Gallbladder mucoceles may result from chronic injury to the epithelial lining of the biliary system since hypersecretion of mucin is the typical physiologic response of any epithelial lining to injury. Recently Shetland Sheepdogs were identified as a breed that is predisposed to gallbladder mucocele formation, suggesting a genetic predisposition (Aguirre et al., J. Am. Vet. Med. Assoc., 231: 79-88, 2007).

Currently, no medical treatment options exist for managing dogs with gallbladder mucoceles; primarily because information regarding the etiology of the disease has been lacking. Additionally, because of the increased frequency and severity of hepatobiliary disease and related conditions in both humans and dogs, there is a pronounced need in the art for novel and efficacious compositions and methods for detection, diagnosis, and treatment of hepatobiliary disease and related conditions in mammals, and in particular in humans an dogs.

### SUMMARY

Disclosed herein are methods of detecting predisposition to or presence of hepatobiliary disease in a canine subject, comprising detecting a nucleotide insertion in the ABCB4 gene, wherein the nucleotide insertion results in premature termination of ABCB4 translation.

Also described are methods of selectively breeding dogs to decrease the frequency of hepatobiliary disease in a dog population, the method comprising identifying dogs in a breeding population that have a predisposition to hepatobiliary disease by the methods disclosed herein; selecting for breeding those dogs that do not have a predisposition to hepatobiliary disease; and breeding only selected dogs, thereby decreasing the frequency of hepatobiliary disease in the dog population.

Further described are kits for detecting the predisposition to or presence of hepatobiliary disease in a canine subject, comprising at least one agent capable of detecting an ABCB4 1583_{—}1584G genotype in the canine subject comprising at least one of (a) an oligonucleotide that specifically hybridizes to a region of ABCB4 DNA comprising an insertion between nucleotides 1582 and 1583 of SEQ ID NO: 1; or (b) an antibody that specifically recognizes a truncated ABCB4 polypeptide set forth as SEQ ID NO: 4 and does not recognize a full-length ABCB4 polypeptide; and instructions for using the agent to detect the ABCB4 1583_{—}1584G genotype.

Additionally described are treatment programs to inhibit or prevent development of gallbladder mucocele in a canine subject, the treatment program comprising detecting a canine subject that is predisposed to or has a hepatobiliary disease comprising a gallbladder mucocele by the methods disclosed herein; and administering to the canine subject bile salts that are more hydrophilic than canine bile salts; thereby inhibiting the development of gallbladder mucocele in the canine subject.

The foregoing and other objects, features, and advantages will become more apparent from the following detailed description, which proceeds with reference to the accompanying figures.

### BRIEF DESCRIPTION OF THE DRAWINGS

FIG. 1 is two DNA sequence chromatograms showing the wildtype and a mutant allele of canine ABCB4. The wildtype sequence (top panel) corresponds to nucleotides 1578 to 1586 of SEQ ID NO: 1. The ABCB4 1583_{—}1584G genotype is an insertion of a guanine between positions 1582 and 1583 of the wildtype cDNA sequence. This insertion is indicated by an arrow in the mutant sequence (bottom panel). This sequence corresponds to positions 1578 to 1587 of SEQ ID NO: 3.

FIG. 2 is two representative gels showing allele-specific PCR amplification of ABCB4 from three affected (diagnosed with gallbladder mucocele) and 3 unaffected Shetland Sheepdogs. Allele-specific primers amplified both wildtype (top panel) and mutant (bottom panel) alleles in affected Shetland Sheepdogs, but only wildtype sequence was amplified in unaffected Shetland Sheepdogs.

FIG. 3 is a pair of schematic drawings showing structural context of the wildtype (FIG. 3A) and ABCB4 1583_{—}1584G mutant (FIG. 3B) proteins in the hepatocyte cell membrane. Also shown in FIGS. 3A and 3B is a schematic representation of the 26 ABCB4 ORFs, showing the location of the ABCB4 1583_{—}1584G insertion mutation and generated stop codons within exon 12.

### SEQUENCE LISTING

The nucleic and/or amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleic acid sequence encoding the wild type canine ABCB4 protein.

SEQ ID NO: 2 is the amino acid sequence of the wild type canine ABCB4 protein.

SEQ ID NO: 3 is the nucleic acid sequence corresponding to the truncated ABCB4 protein resulting from a guanine insertion between positions 1582 and 1583 of the nucleic acid sequence encoding the wildtype ABCB4 protein. The insertion produces a frame-shift that produces four premature stop codons in exon 12.

SEQ ID NO: 4 is the amino acid sequence of the truncated ABCB4 protein resulting from a guanine insertion at position 1583 in SEQ ID NO: 3.

SEQ ID NOs: 5 and 6 are the nucleic acid sequences of representative forward and reverse allele-specific PCR primers used to amplify and identify the presence of the mutant ABCB4 1583_{—}1584G allele. SEQ ID NO: 6 is also used as a reverse primer to amplify the wildtype ABCB4 allele.

SEQ ID NOs: 7 is the nucleic acid sequence of a representative forward allele-specific PCR primer used to amplify and detect the presence of the wild type ABCB4 allele.

SEQ ID NOs: 8-59 are the nucleic acid sequences for representative forward and reverse PCR primers used to amplify ABCB4 exons 1-26 as detailed in Table 1.

### DETAILED DESCRIPTION

### I. Abbreviations

ASOH allele-specific oligonucleotide hybridization

cDNA complementary DNA

DASH dynamic allele-specific hybridization

ELISA enzyme-linked immunosorbant assay

GM gallbladder mucocele

IF immunofluorescence

Mb megabase

OLA oligonucleotide ligation assay

ORF open reading frame

PC phosphatidylcholine

PCR polymerase chain reaction

RT-PCR reverse transcription polymerase chain reaction

SNP single nucleotide polymorphism

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

ABCB4: An ATP binding cassette lipid translocator. ABCB4 is expressed on canalicular membranes of hepatocytes where it translocates phosphatidylcholine (PC) from the hepatocyte to the biliary canalicular lumen. The gene and gene product names “ABCB4” and “MDR3” are used equivalently in the art. As described herein, the ABCB4 1583_{—}1584G genotype is indicative of gallbladder mucocele in dogs.

Altered expression: Expression of a biological molecule (for example, mRNA or protein) in a subject or biological sample from a subject that deviates from expression of the same biological molecule in a subject or biological sample from a subject having normal or unaltered characteristics for the biological condition associated with the molecule. Aberrant expression is synonymous with altered expression. Normal expression can be found in a control, a standard for a population, etc. Altered or aberrant expression of a biological molecule may be associated with a disease. The term “associated with” includes an increased risk of developing the disease as well as the disease itself. Expression may be altered in such a manner as to be increased or decreased. The directed alteration in expression of mRNA or protein may be associated with therapeutic benefits. In particular examples, altered expression of a gene, for example the ABCB4 gene, is a result of a defect in one or both copies of that gene in the genome of a subject.

Altered protein expression refers to expression of a protein that is in some manner different from expression of the protein in a normal (wild type) situation. This includes but is not necessarily limited to: (1) a mutation in the protein such that one or more of the amino acid residues is different; (2) a short deletion or addition of one or a few amino acid residues to the sequence of the protein; (3) a longer deletion or addition of amino acid residues, such that an entire protein domain or sub-domain is removed or added; (4) expression of an increased amount of the protein, compared to a control or standard amount; (5) expression of an decreased amount of the protein, compared to a control or standard amount; (6) alteration of the subcellular localization or targeting of the protein; (7) alteration of the temporally regulated expression of the protein (such that the protein is expressed when it normally would not be, or alternatively is not expressed when it normally would be); and (8) alteration of the localized (for example, organ or tissue specific) expression of the protein (such that the protein is not expressed where it would normally be expressed or is expressed where it normally would not be expressed), each compared to a control or standard. In particular examples, the expression of the ABC4 protein is altered as the result of a frameshift insertion mutation in exon 12 of the ABCB4 gene that produces a truncated ABCB4 protein.

Amplify: Increase the number or amount of something, such as a molecule or compound. To amplify a molecule of DNA is to increase the copy number of the particular DNA molecule, e.g., through an in vitro amplification technique. DNA can be amplified by any method that replicates the DNA sequence and increases the copy number of that sequence. In particular examples, DNA amplification is achieved in some embodiments using a PCR-based method including RT-PCR. Other exemplary methods of DNA amplification include isothermal amplification methods. Other representative and non-limiting examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one light (about 25 kD) and one heavy chain (about 50-70 kD). The N-terminus of each chain defines a variable region of about 100 to 110 or more amino acids primarily responsible for antigen recognition. The terms variable light chain (V_{L}) and variable heavy chain (V_{H}) refer, respectively, to these light and heavy chains.

As used herein, the term antibody includes intact immunoglobulins as well as a number of well-characterized fragments produced by digestion with various peptidases, or genetically engineered artificial antibodies. Thus, for example, pepsin digests an antibody below the disulfide linkages in the hinge region to produce F(ab)′₂, a dimer of Fab which itself is a light chain joined to V_{H}-C_{H} by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab with part of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de novo either chemically or by utilizing recombinant DNA methodology. Thus, the term antibody as used herein also includes antibody fragments either produced by the modification of whole antibodies or synthesized de novo using recombinant DNA methodologies.

Antibodies for use in the methods of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988).

The terms bind specifically and specific binding refer to the ability of a specific binding agent (such as, an antibody) to bind to a target molecular species in preference to binding to other molecular species with which the specific binding agent and target molecular species are admixed. A specific binding agent is said specifically to recognize a target molecular species when it can bind specifically to that target.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988). Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994). A chimeric antibody is an antibody that contains one or more regions from one antibody and one or more regions from one or more other antibodies. An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Bile salts: Component of bile that is produced by the liver and functions in digestion and absorption of dietary fat. Bile salts form simple micelles in the absence of phospholipids such as PC and mixed micelles with PC, when PC is present. As a detergent, bile salts are generally cytotoxic in the simple micelle form. The degree of cytotoxicity increases with the hydrophobicity of the bile salts. Bile salt hydrophobicity varies with the species of bile-salt producing animal. For example, human bile salts (chemodeoxycholate) are more hydrophobic and more cytotoxic than bear bile salts (ursodeoxycholate) and mouse bile salts. Canine bile salts (cholate) are generally closer to the human type in hydrophobicity and cytotoxicity. The anion form of a bile salt is called a bile acid—generally the term bile salt and bile acid are used interchangeably.

Breeding population: A population of dogs who are potentially suitable for breeding (producing successive generations of dogs). A breeding population can be of any number of dogs, though it must include at least one male and one female dog.

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA extracted from cells.

Contacting: “Contacting” includes in solution and solid phase, for example contacting cells or other biological sample with a test compound. In one example, contacting includes contacting a population of cells isolated from an hepatobiliary disease-affected dog.

Control: A “control” refers to a sample or standard used for comparison with a test sample. In some embodiments, the control is a sample obtained from a healthy subject that does not have symptoms of or a predisposition to a disease such as a hepatobiliary disease. In some embodiments, the control is a historical control or standard reference value or range of values (such as a previously tested control sample, such as a group of hepatobiliary disease-unaffected subjects, or group of samples that represent baseline or normal values, such as the normal level of wild type ABCB4 protein expression in a hepatobiliary disease-unaffected subject). Control standards and values may be set based on a known or determined population value and may be supplied in the format of a graph or table that permits easy comparison of measured, experimentally determined values.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. The determination of a given order of nucleotides in a DNA of interest is referred to as DNA sequencing.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Deletion: The removal of a sequence of DNA (which may be as short as a single nucleotide), the regions on either side being joined together. A deletion in a DNA sequence is a genetic defect that can be directly detected by sequencing the region of DNA encompassing the site of deletion, thereby detecting the absence of the particular deleted sequence.

Frequency: A measure of the incidence of a disease in a population. Disease frequency is determined by any method known to the art of diagnosing the particular disease in the population. In particular examples, the frequency of hepatobiliary disease in a dog population can be determined by detecting the herein-described insertion in exon 12 of the ABCB4 gene.

Gallbladder mucoceles: A hepatobiliary disease characterized by abnormal secretion of mucin into the gallbladder, thereby producing organ distensions. Gallbladder mucocele is also known as mucinous hyperplasia or mucinous cholecystitis. Gallbladder mucocele is relatively uncommon in humans, but is an increasingly diagnosed adult-onset canine disease.

Gene expression: The process by which the coded information of a nucleic acid transcriptional unit (including, for example, genomic DNA or cDNA) is converted into an operational, non-operational, or structural part of a cell, often including the synthesis of a protein. Gene expression can be influenced by external signals; for instance, exposure of a subject to an agent that inhibits gene expression. Expression of a gene also may be regulated anywhere in the pathway from DNA to RNA to protein. Regulation of gene expression occurs, for instance, through controls acting on transcription, translation, RNA transport and processing, degradation of intermediary molecules such as mRNA, or through activation, inactivation, compartmentalization or degradation of specific protein molecules after they have been made, or by combinations thereof. Gene expression may be measured at the RNA level or the protein level and by any method known in the art, including Northern blot, RT-PCR, primer extension, Western blot, immunofluorescent or immunocytochemical protein detection, or in vitro, in situ, or in vivo protein activity assay(s).

The expression of a nucleic acid may be modulated compared to a control state, such as at a control time (for example, prior to administration of a substance or agent that affects regulation of the nucleic acid under observation) or in a control cell or subject, or as compared to another nucleic acid. Such modulation includes but is not necessarily limited to overexpression, underexpression, or suppression of expression. In addition, it is understood that modulation of nucleic acid expression may be associated with, and in fact may result in, a modulation in the expression of an encoded protein or even a protein that is not encoded by that nucleic acid.

Expression of a target gene may be measured by any method known to those of skill in the art, including for example measuring mRNA or protein levels. It is understood that a measurable reduction in gene expression is relative, and does not require absolute suppression of the gene. A measurable reduction in gene expression is a test sample only requires that gene expression is measurably less than a control. Thus, in certain embodiments, a measurable reduction in gene expression requires that the gene is expressed at least 5% less than control expression levels, or at least 10% less, at least 15% less, at least 20% less, at least 25% less, or even more reduced from control levels. Thus, in some particular embodiments, a measurable reduction in gene expression is about 30%, about 40%, about 50%, about 60%, or more reduced from control levels. In specific examples, expression is reduced by 70%, 80%, 85%, 90%, 95%, or even more. Gene expression is substantially eliminated when expression of the gene is reduced by 90%, 95%, 98%, 99% or even 100%.

Hepatobiliary disease: General category of diseases resultant from abnormalities in the biliary system. Common forms of hepatobiliary disease associated with ABCB4 dysfunction include progressive familial intrahepatic cholestasis (type 3), cholelithiasis, primary biliary cirrhosis, and intrahepatic cholestasis of pregnancy. Described herein is the previously-unknown association between gallbladder mucocele and ABCB4 dysfunction. In humans and dogs, the severity of the disease can range from fatal (without a liver transplant) to milder forms. In severely affected people, the disease manifests during early childhood and is fatal without a liver transplant.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as base pairing. More specifically, A will hydrogen bond to T or U, and G will bond to C. Complementary refers to the base pairing that occurs between to distinct nucleic acid sequences or two distinct regions of the same nucleic acid sequence.

Isolated: An isolated biological component (such as a nucleic acid, peptide or protein) has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been isolated thus include nucleic acids and proteins purified by standard purification methods. The term also embraces nucleic acids, peptides and proteins prepared by recombinant expression in a host cell as well as chemically synthesized nucleic acids. The terms isolated and purified do not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation.

Label: A detectable compound or composition that is conjugated directly or indirectly to another molecule to facilitate detection of that molecule. Specific, non-limiting examples of labels include fluorescent tags, enzymatic linkages, and radioactive isotopes.

Mutation: Any change of DNA sequence, for instance within a gene or chromosome. As used herein, the term “genetic defect” is synonymous with “mutation”. In some instances, a mutation will alter a characteristic or trait (phenotype), but this is not always the case. Types of mutations include base substitution point mutations (for example, transitions or transversions), deletions, and insertions. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

This term also encompasses DNA alterations that are present constitutionally, that alter the function of the encoded protein in a readily demonstrable manner, and that can be inherited by the children of an affected individual.

Gene mutations that occur outside of amino acid coding regions (i.e. untranslated regions) may also affect gene expression by alerting the binding sites for transcription factors on the DNA.

Nucleic acid molecule: A polymeric form of nucleotides, which may include both sense and anti-sense strands of ribonucleic acid (RNA), cDNA, genomic DNA, and synthetic forms and mixed polymers thereof. A nucleotide refers to a ribonucleotide, deoxynucleotide or a modified form of either type of nucleotide. A nucleic acid molecule as used herein is synonymous with nucleic acid and polynucleotide. A nucleic acid molecule is usually at least 10 bases in length, unless otherwise specified. The term includes single- and double-stranded forms. A polynucleotide may include either or both naturally occurring and modified nucleotides linked together by naturally occurring and/or non-naturally occurring nucleotide linkages.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term nucleic acid molecule also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, the left hand end of a polynucleotide sequence written in the sense orientation is the 5′ end and the right hand end of the sequence is the 3′ end. In addition, the left hand direction of a polynucleotide sequence written in the sense orientation is referred to as the 5′ direction, or upstream, while the right hand direction of the polynucleotide sequence is referred to as the 3′ direction, or downstream. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules and morpholinos.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases.

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell.

Polymerase Chain Reaction (PCR): An in vitro amplification technique that increases the number of copies of a nucleic acid molecule (for example, a nucleic acid molecule in a sample or specimen). The product of a PCR can be characterized by standard techniques known in the art, such as electrophoresis, restriction endonuclease cleavage patterns, oligonucleotide hybridization or ligation, and/or nucleic acid sequencing.

In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand). Primers can be selected that include at least 15, at least 20, at least 25, at least 30, at least 35, at least 40, at least 45, at least 50 or more consecutive nucleotides of a target nucleotide sequence. In particular examples, primers are used as pairs. In some examples, at least one of the primers used in a PCR amplification reaction are allele-specific, and will selectively bind to, and amplify a particular allele of a gene.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

Polymorphism: Variant in a sequence of a gene, usually carried from one generation to another in a population. Polymorphisms can be those variations (nucleotide sequence differences) that, while having a different nucleotide sequence, produce functionally equivalent gene products, such as those variations generally found between individuals, different ethnic groups, geographic locations. The term polymorphism also encompasses variations that produce gene products with altered function, i.e., variants in the gene sequence that lead to gene products that are not functionally equivalent. This term also encompasses variations that produce no gene product, an inactive gene product, or increased or increased activity gene product.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation (e.g., an alteration of a secondary structure such as a stem-loop, or an alteration of the binding affinity of the nucleic acid for associated molecules, such as polymerases, RNases, and so forth).

Predisposition to a disease: To be susceptible and prone to a disease. A genetic defect that results in (or causes) a disease can make a subject “predisposed” to the particular disease, though the subject may not yet exhibit physical symptoms of the disease. Such a subject may become symptomatic over time or upon exposure to certain environmental or other stimulus. In particular examples, dogs carrying a genetic defect in the ABCB4 gene that do not show symptoms of hepatobiliary disease (e.g., evidence of gallbladder mucocele prior to late adulthood) can be considered to be predisposed to hepatobiliary disease prior to symptom presentation.

Preventing or treating a disease: Preventing a disease refers to inhibiting the full development of a disease, for example inhibiting the development of myocardial infarction in a person who has coronary artery disease, inhibiting the progression or metastasis of a tumor in a subject with a neoplasm, or inhibiting formation of gallbladder mucoceles in a subject with hepatobiliary disease. Treatment refers to a therapeutic intervention that ameliorates a sign or symptom of a disease or pathological condition after it has begun to develop.

Protein: A biological molecule, particularly a polypeptide, expressed by a gene and comprised of amino acids.

Purified: In a more pure form than is found in nature. The term purified does not require absolute purity; rather, it is intended as a relative term. Thus, for example, a purified protein preparation is one in which the protein referred to is more pure than the protein in its natural environment within a cell.

The term substantially purified as used herein refers to a molecule (for example, a nucleic acid, polypeptide, oligonucleotide, etc.) that is substantially free of other proteins, lipids, carbohydrates, or other materials with which it is naturally associated. In one embodiment, a substantially purified molecule is a polypeptide that is at least 50% free of other proteins, lipids, carbohydrates, or other materials with which it is naturally associated. In another embodiment, the polypeptide is at least at least 80% free of other proteins, lipids, carbohydrates, or other materials with which it is naturally associated. In yet other embodiments, the polypeptide is at least 90% or at least 95% free of other proteins, lipids, carbohydrates, or other materials with which it is naturally associated.

Quantitative real time PCR: A method for detecting and measuring products generated during each cycle of a PCR, which products are proportionate to the amount of template nucleic acid present prior to the start of PCR. The information obtained, such as an amplification curve, can be used to quantitate the initial amounts of template nucleic acid sequence.

Reverse-transcription PCR (RT-PCR): A method for detecting, quantifying, or utilizing RNA present in a sample by a procedure wherein the RNA serves as a template for the synthesis of cDNA by a reverse transcriptase followed by PCR to amplify the cDNA. RT-PCR can be used in combination with quantitative real time PCR as a method of measuring the quantity of starting template in the reaction.

RNA (ribonucleic acid): A typically linear polymer of ribonucleic acid monomers, linked by phosphodiester bonds. Naturally occurring RNA molecules fall into three general classes, messenger (mRNA, which encodes proteins), ribosomal (rRNA, components of ribosomes), and transfer (tRNA, molecules responsible for transferring amino acid monomers to the ribosome during protein synthesis). Messenger RNA includes heteronuclear (hnRNA) and membrane-associated polysomal RNA (attached to the rough endoplasmic reticulum). Total RNA refers to a heterogeneous mixture of all types of RNA molecules.

Sample (or biological sample): A biological specimen containing genomic DNA, RNA (including mRNA), protein, or combinations thereof, obtained from a subject. Examples include, but are not limited to, peripheral blood or a fraction thereof, fine needle aspirate, urine, saliva, cheek swab, tissue biopsy, surgical specimen, and autopsy material.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. The differences between two nucleic acid sequences can also be referred to as sequence “variants”.

Methods of alignment of sequences for comparison are well known in the art. Various programs and alignment algorithms are described in: Smith and Waterman (Adv. Appl. Math. 2: 482, 1981); Needleman and Wunsch (J. Mol. Biol., 48: 443, 1970); Pearson and Lipman (PNAS USA, 85:2444, 1988); Higgins and Sharp (Gene, 73:237-244, 1988); Higgins and Sharp (CABIOS 5:151-153, 1989); Corpet et al. (Nuc. Acids Res. 16:10881-10890, 1988); Huang et al. (Comp. Appls Biosci. 8:155-165, 1992); and Pearson et al. (Meth. Mol. Biol. 24:307-31, 1994). Altschul et al. (Nature Genet., 6:119-129, 1994) presents a detailed consideration of sequence alignment methods and homology calculations.

An alternative indication that two nucleic acid molecules are closely related is that the two molecules specifically hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (In Molecular Cloning: A Laboratory Manual, Cold Spring Harbor, N.Y., 1989) and Tijssen (Laboratory Techniques in Biochemistry and Molecular Biology Part I, Ch. 2, Elsevier, New York, 1993).

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference. The following is an exemplary set of hybridization conditions:

Very High Stringency (Detects Sequences that Share 90% Identity or Greater)

| | |
|---|---|
| Hybridization: | 5x SSC at 65° C. for 16 hours |
| Wash twice: | 2x SSC at room temperature (RT) |
| | for 15 minutes each |
| Wash twice: | 0.5x SSC at 65° C. for 20 minutes each |

High Stringency (Detects Sequences that Share 80% Identity or Greater)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share 50% Identity or Greater)

| | |
|---|---|
| Hybridization: | 6x SSC at RT to 55° C. for 16-20 hours |
| Wash at | 2x-3x SSC at RT to 55° C. for |
| least twice: | 20-30 minutes each. |

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specifically hybridizable and specifically complementary are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Subject: Living multi-cellular vertebrate organism, a category that includes human and non-human mammals. In particular examples, the subject is a canine subject. In other examples, the canine subject is a particular breed of dog, such as a Shetland sheepdog.

Therapeutic: A generic term that includes both diagnosis and treatment.

Therapeutically effective amount: A quantity of compound sufficient to achieve a desired effect in a subject being treated.

An effective amount of a compound may be administered in a single dose, or in several doses, for example daily, during a course of treatment. However, the effective amount will be dependent on the compound applied, the subject being treated, the severity and type of the affliction, and the manner of administration of the compound. For example, a therapeutically effective amount of an active ingredient can be measured as the concentration (moles per liter or molar-M) of the active ingredient (such as a small molecule, peptide, protein, or antibody) in blood (in vivo) or a buffer (in vitro) that produces an effect.

Treating a disease: Includes inhibiting or preventing the partial or full development or progression of a disease, for example in an animal or person who is known to have a predisposition to a disease. Furthermore, treating a disease refers to a therapeutic intervention that ameliorates at least one sign or symptom of a disease or pathological condition, or interferes with a pathophysiological process, after the disease or pathological condition has begun to develop.

In particular examples, treating a hepatobiliary disease such as gallbladder mucocele involves supplementing the diet of afflicted canine subjects with bile salts such as ursodeoxycholate that are less cytotoxic than native canine bile salts.

Under conditions sufficient for: A phrase that is used to describe any environment that permits the desired activity.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Disclosed herein are methods of detecting predisposition to or presence of hepatobiliary disease in a canine subject, comprising detecting a nucleotide insertion in the ABCB4 gene, wherein the nucleotide insertion results in premature termination of ABCB4 translation. In particular examples, the nucleotide insertion is located within or 5′ (upstream) of exon 12 of the ABCB4 gene, for example between nucleotides 1582 and 1583 of ABCB4 nucleic acid sequence as set forth as SEQ ID NO: 1. In some examples, the canine subject is selected from the group consisting of Shetland Sheepdog, Cairn Terrier, Cocker Spaniel, and Pomeranian. In further examples, the hepatobiliary disease comprises gallbladder mucocele. In some examples, detecting the nucleotide insertion in the ABCB4 gene comprises isolating DNA from the canine subject; and sequencing a region of the DNA including the nucleotide sequence corresponding to nucleotides 1582-1583 of SEQ ID NO: 1, and in further examples, can comprise amplifying the region of the DNA including the nucleotide sequence corresponding to nucleotides 1582-1583 of SEQ ID NO: 1, prior to sequencing. In other examples, detecting the nucleotide insertion in the ABCB4 gene comprises allele-specific PCR comprising isolating DNA from the canine subject; contacting the DNA with a primer pair comprising a forward primer and a reverse primer under conditions sufficient to amplify ABCB4 DNA, wherein either the forward or the reverse primer comprises an oligonucleotide that specifically hybridizes to a region of the ABCB4 DNA comprising the nucleotide insertion; and detecting amplified ABCB4 DNA comprising the nucleotide insertion. In yet further examples, the forward primer comprises an oligonucleotide comprising a sequence no more than three nucleotides variant from SEQ ID NO: 5, except the G at position 24 is invariant, and wherein the reverse primer comprises an oligonucleotide comprising a sequence no more than three nucleotides variant from SEQ ID NO: 6.

Also described are methods of selectively breeding dogs to decrease the frequency of hepatobiliary disease in a dog population, the method comprising identifying dogs in a breeding population that have a predisposition to hepatobiliary disease by the methods disclosed herein of detecting a nucleotide insertion in the ABCB4 gene; selecting for breeding those dogs that do not have a predisposition to hepatobiliary disease; and breeding only selected dogs, thereby decreasing the frequency of hepatobiliary disease in the dog population. In particular examples the nucleotide insertion is located between nucleotides 1582 and 1583 of ABCB4 sequence as set forth as SEQ ID NO: 1. In some examples, the canine subject is selected from the group consisting of Shetland Sheepdog, Cairn Terrier, Cocker Spaniel, and Pomeranian. In further examples, the hepatobiliary disease comprises gallbladder mucocele. In particular examples, detecting the nucleotide insertion in the ABCB4 gene comprises isolating DNA from the canine subject; and sequencing a region of the DNA including the nucleotide sequence corresponding to nucleotides 1582-1583 of SEQ ID NO: 1. In other particular examples, detecting the nucleotide insertion in the ABCB4 gene comprises allele-specific PCR comprising isolating DNA from the canine subject; contacting the DNA with a primer pair comprising a forward primer and a reverse primer under conditions sufficient to amplify ABCB4 DNA, wherein either the forward or the reverse primer comprises an oligonucleotide that specifically hybridizes to a region of the ABCB4 DNA comprising the nucleotide insertion; and detecting amplified ABCB4 DNA comprising the nucleotide insertion.

Further described herein are kits for detecting the predisposition to or presence of hepatobiliary disease, for example gallbladder mucocele, in a canine subject, comprising at least one agent capable of detecting an ABCB4 1583_{—}1584G genotype in the canine subject comprising at least one of (a) an oligonucleotide that specifically hybridizes to a region of ABCB4 DNA comprising an insertion between nucleotides 1582 and 1583 of SEQ ID NO: 1; or (b) an antibody that specifically recognizes a truncated ABCB4 polypeptide set forth as SEQ ID NO: 4 and does not recognize a full-length ABCB4 polypeptide; and instructions for using the agent to detect the ABCB4 1583_{—}1584G genotype. In particular examples, the oligonucleotide comprises a sequence no more than three nucleotides variant from SEQ ID NO: 5, except the G at position 24 is invariant.

Additionally described are treatment programs to inhibit or prevent development of gallbladder mucocele in a canine subject, such as a canine subject no older than 1, 2, 3, 4, 5, 6 or 7 years old, the treatment program comprising detecting a canine subject that is predisposed to or has a hepatobiliary disease comprising a gallbladder mucocele by the methods disclosed herein; and administering to the canine subject bile salts that are more hydrophilic than canine bile salts; thereby inhibiting the development of gallbladder mucocele in the canine subject.

### IV. Hepatobiliary Disease and ABCB4

ABCB4 dysfunction has long been associated with hepatobiliary disease in humans and mice. Over three dozen disease-causing mutations in human ABCB4 have been described (Davit-Spraul et al., Orphanet. J. Rare Dis., 4: 1, 2009, Delaunay et al., Hepatology, 49: 1218-1227, 2009, Gonzales et al, Front Biosci., 14: 4242-4256, 2009, and Trauner et al., Semin. Liver Dis., 27: 77-98, 2007). The resultant hepatobiliary diseases range from severe (debilitating diseases of young children that require lever transplantation) to mild. Often disease severity depends on the nature of the mutation. Milder disease occurs when the ABCB4 gene mutation reduces but does not eliminate transport activity of the protein. Similarly, milder forms of disease exist in patients that are heterozygous for mutations that eliminate transporter activity (i.e., truncations). Abcb4−/− mice also develop biliary disease. Despite the known association between ABCB4 mutation and hepatobiliary disease in humans and mice, no such association has previously been identified in dogs.

Gallbladder mucocele is an autosomal dominant hepatobiliary disease (with incomplete penetrance) more prevalent in dogs than other species. Though inherited, gallbladder mucocele symptoms are adult-onset. Thus, as with many genetic disorders, prior to the onset of symptoms, subjects carrying a causative genetic defect can be considered “predisposed” to the disease. The median age of Shetland Sheepdogs diagnosed with gallbladder mucocele reported in one study was 10.9 years of age (Aguirre et al., J. Am. Vet. Med. Assoc., 231: 79-88, 2007), while that identified in the study population described herein was 9 years of age. The median age of the other affected dogs described herein was 11 years of age.

Prior to the current disclosure, gallbladder mucocele were generally diagnosed by ultrasonography following symptoms such as abdominal distension, sensitivity and fever. Despite surgical intervention, many dogs are severely affected by the time of diagnosis and the disease is often fatal.

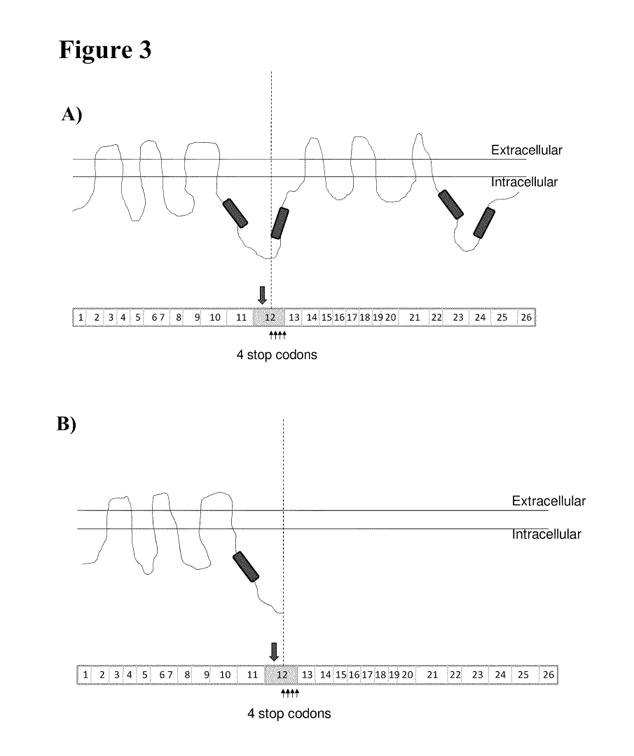

Disclosed herein, is the identification of an insertion (G) mutation in exon 12 of the canine ABCB4 gene that is associated with canine gallbladder mucocele. This insertion results in a frameshift in translation of the transcribed ABCB4 sequence, generating four stop codons that prematurely terminate ABCB4 protein synthesis within exon 12, producing a truncated ABCB4 missing over half of the protein including critical ATP binding sites and a putative substrate (PC) binding site (see FIG. 3 for a schematic illustration). As shown herein, a significant association (P=1.54E-7) was detected between affected Shetland Sheepdogs and the insertion mutation. The identical mutation was identified in three other dogs with gallbladder mucoceles (Pomeranian, Cairn Terrier, and Cocker Spaniel). Thus, ABCB4 appears to play a key role in hepatobiliary health in dogs as it does in people. Affected dogs may provide a useful model for identifying novel treatment strategies for ABCB4-associated biliary disease in people. The etiology of gallbladder mucoceles in dogs is currently unknown, but the results reported herein provide definitive evidence that ABCB4 is involved. Hepatocyte PC transport, and therefore bile PC content, in affected dogs identified in this study would, therefore, be decreased compared to wildtype dogs. Biliary epithelial lining cells would be subjected to bile salt-induced injury because of diminished ability to form mixed micelles (Anwer et al., Vet. Clin. North Am. Small Anim. Pract., 25: 503-517, 1995). A common physiologic response of epithelial linings to injury is mucinous hyperplasia, a histopathologic finding frequently described in dogs diagnosed with gallbladder mucocele. Furthermore, exposure to bile salts has been shown to stimulate mucin secretion in cultured canine gallbladder epithelial cells (Klinkspoor et al., Biochem. J., 332, (Pt 1): 257-262, 1998). Thus, gallbladder epithelium in dogs that harbor ABCB4 1583_{—}1584G would be exposed to greater concentrations of unbuffered bile salts than wildtype dogs, resulting in greater mucin secretion, mucinous hyperplasia, and eventually mucocele formation.

Significantly, the present discovery of an insertion mutation in canine ABCB4 allows early identification of dogs predisposed to gallbladder mucocele formation. According to additional aspects, this creates a number of beneficial applications for dogs as well as potential benefits for people. For example, in certain aspects, genotyping of young dogs for ABCB4 1583_{—}1584G allows veterinarians to closely monitor gallbladder mucocele development and progression in affected dogs.

In further aspects, surgical intervention could be performed earlier in the disease process before disease-induced morbidity places the patient at higher risk for intra- and post-operative complications.

Currently, no medical treatment options exist for managing dogs with gallbladder mucoceles primarily because information regarding the etiology of the disease has been lacking. Some human patients with ABCB4-associated biliary disease benefit from treatment with ursodeoxycholate, a hydrophilic and much less cytotoxic bile acid than the dominant endogenous bile salt chenodeoxycholate (Oude Elferink et al., Pflugers Arch, 453: 601-610, 2007). According to additional aspects, dogs with the ABCB4 1583_{—}1584G mutation provide a model for the various biliary diseases in people that result from similar ABCB4 mutations, and can be used for testing potential human treatment regimes.

The occurrence of gallbladder mucoceles in Shetland Sheepdogs appears to be inherited in a dominant fashion with incomplete penetrance. No dogs in this study population were homozygous for the mutation. Because a more severe phenotype is observed in people homozygous for mutations resulting in elimination of ABCB4 protein function, likely the same would be true for dogs. In severely affected people, the disease manifests during early childhood and is fatal without a liver transplant (Oude Elferink et al., Pflugers Arch, 453: 601-610, 2007). It is possible that homozygosity for the mutation results in death of affected dogs in early puppyhood.

### V. Molecular Methods of Diagnosing Canine Hepatobiliary Disease

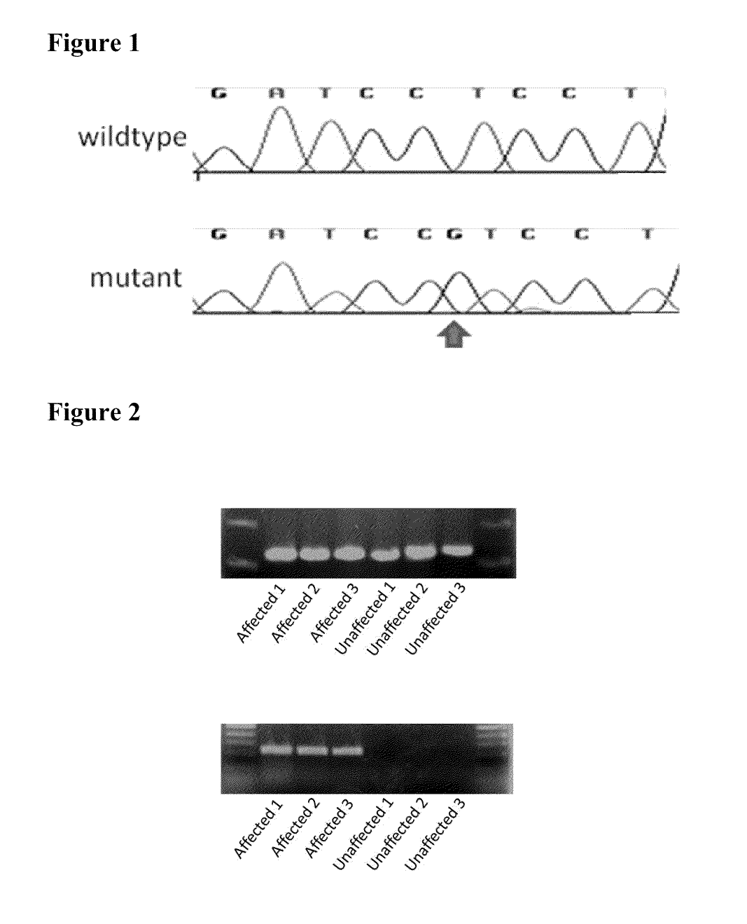

The determination that gallbladder mucocele in dogs is caused by a single base insertion in exon 12 of the ABCB4 gene (located on chromosome 14) enables molecular methods of gallbladder mucocele diagnosis that are not reliant on gallbladder mucocele symptom onset. The inserted nucleotide, which is shown in FIG. 1 (indicated by the arrow) corresponds to a G insertion between positions 1582 and 1583 of SEQ ID NO: 1. For reference, the entire sequence of canine Chromosome 14 is incorporated herein by reference based on the publicly available canine genomic DNA sequence from GenBank (accession number NC_{—}006596.2) as of Aug. 13, 2009. The canine genomic sequence can also be viewed at the USCS Genome Bioinformatics site, available on-line at genome.ucsc.edu/cgi-bin/hgGateway?org=Dog&db=canFam2&hgsid=156525325. The G insertion in exon 12 is a frameshift mutation that introduces four premature stop codons shortly after the insertion and truncates the translated ABCB4 protein by approximately 50% (see FIG. 3). Thus, any defect in the ABCB4 gene within and upstream of exon 12 that similarly truncates the ABCB4 protein will likely have a similar association with canine hepatobiliary disease.

In view of the teachings provided herein, it will be appreciated that a genetic defect in the ABCB4 gene that results in gallbladder mucocele is detectable by any method of detecting an insertion in the ABCB4 gene, and it will be further appreciated that detection of the genetic defect is a suitable diagnostic test for hepatobiliary disease, and particularly gallbladder mucocele in a dog. Moreover, it will also be understood that identification of the ABCB4 genetic defect in a dog prior to the development of gallbladder mucocele enables proactive treatment of the disease-predisposed dog for instance with dietary supplement of less cytotoxic bile salts that minimize or even eliminate the development of gallbladder mucocele. Additionally, information regarding a dog's ABCB4 genotype (unaffected, heterozygous or homozygous) as indicated, for example, by the presence of the ABCB4 1583_{—}1584G genotype can be tested early in the life of the individual dog, and optionally included on a license, medical record, pedigree, and so forth. Such information can also be used in making breeding decisions with regard to the tested individual, and decrease the frequency of gallbladder mucocele in a dog population.

The ABCB4 1583_{—}1584G genotype is prevalent in gallbladder mucocele-affected Shetland Sheepdogs. As described herein, an association between gallbladder mucocele and the ABCB4 mutant genotype was also observed in Cairn Terrier, Cocker Spaniel, and Pomeranian dogs. Thus, it will be appreciated that the presence of the ABCB4 1583_{—}1584G genotype will be indicative of gallbladder mucocele in any dog. This genotype can be determined directly (i.e. through detection of the inserted nucleotide in exon 12) or indirectly, such as through detection of the truncated ABCB4 protein. Other indirect methods of detection of the ABCB4 1583_{—}1584G genotype include measurement of PC concentration in the bile of a subject. In particular examples, PC is aspirated from a subject by ultrasound and measured by any method known to the art.

### A. Detection of an Insertion in Exon 12 of ABCB4

In one embodiment of the disclosed methods, gallbladder mucocele-affected dogs are identified by the direct detection of an insertion in exon 12 of the ABCB4 gene. In particular examples, the insertion is a guanine nucleotide between positions 1582 and 1583 (referred to as the ABCB4 1583_{—}1584G genotype). To perform a diagnostic test for the presence or absence of the ABCB4 1583_{—}1584G genotype (or another similar insertional mutation), a suitable genomic DNA-containing sample from a subject is obtained and the DNA extracted using conventional techniques. DNA is extracted from any suitable biological sample, for example, a blood sample, a buccal swab, a hair follicle preparation, or a nasal aspirate is used as a source of cells to provide the DNA sample. In a particular embodiment, the extracted DNA is then subjected to amplification of some or all of the ABCB4 sequence, for example according to standard procedures. The presence or absence of the ABCB4 1583_{—}1584G genotype is determined, for instance by sequencing (directly or indirectly) a region of DNA encompassing the chromosomal location of the ABCB4 1583_{—}1584G genotype. Conventional DNA sequencing methods may be used, such as manual and automated fluorescent DNA sequencing, primer extension methods (Nikiforov et al., Nucl. Acids Res., 22:4167-4175, 1994), allele-specific PCR methods (Rust et al., Nucl. Acids Res., 6:3623-3629, 1993), RNase mismatch cleavage, single-strand conformation polymorphism (SSCP), denaturing gradient gel electrophoresis (DGGE), oligonucleotide hybridization, and the like. Also, see the following U.S. patents for descriptions of methods or applications of polymorphism analysis to disease prediction and/or diagnosis: U.S. Pat. Nos. 4,666,828; 4,801,531; 5,110,920; 5,268,267; and 5,387,506.

In some embodiments, sequence surrounding and overlapping the insertion between ABCB4 positions 1583 and 1584, such as sequences surrounding this region in SEQ ID NO: 1 or SEQ ID NO: 3, can be useful for a number of gene mapping, targeting, and detection procedures. For example, genetic probes can be readily prepared for hybridization and detection of the described insertion. Such probe sequences may be greater than about 12 or more oligonucleotides in length and possess sufficient complementarity to distinguish between a wild-type and a ABCB4 1583_{—}1584G genotype sequence. Similarly, sequences surrounding and overlapping the specifically disclosed insertion can be utilized in allele-specific hybridization procedures.

In some embodiments, the ABCB4 1583_{—}1584G genotype can be detected by allele-specific oligonucleotide hybridization (ASOH) (Stoneking et al., Am. J. Hum. Genet. 48:370-382, 1991), which involves hybridization of labeled oligonucleotide probes to the sequence, stringent washing, and signal detection. In other embodiments, applicable methods include techniques that incorporate more robust scoring of hybridization. Examples of these procedures include the ligation chain reaction (ASOH plus selective ligation and amplification), as disclosed in Wu and Wallace (Genomics 4:560-569, 1989); mini-sequencing (ASOH plus a single base extension) as discussed in Syvanen (Meth. Mol. Biol. 98:291-298, 1998); and the use of DNA chips (miniaturized ASOH with multiple oligonucleotide arrays) as disclosed in Lipshutz et al. (BioTechniques 19:442-447, 1995). Alternatively, ASOH with single- or dual-labeled probes can be merged with PCR, as in the 5′-exonuclease assay (Heid et al., Genome Res. 6:986-994, 1996), or with molecular beacons (as in Tyagi and Kramer, Nat. Biotechnol. 14:303-308, 1996). Allele-specific PCR can also be used such that DNA comprising the ABCB4 insertion will be amplified using allele-specific primers, but the wild type allele will not be amplified.

The presence of the ABCB4 1583_{—}1584G genotype can also be detected by dynamic allele-specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probe-target duplex) present. The sample is then steadily heated while fluorescence is continually monitored. A rapid fall in fluorescence indicates the denaturing temperature of the probe-target duplex. Using this technique, a single-base mismatch between the probe and target results in a significant and readily detectable lowering of melting temperature (Tₘ).

A variety of other techniques can be used to detect the ABCB4 1583_{—}1584G genotype in the canine DNA. Merely by way of example, see U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for such methods.

### B. Detection of a Truncated ABCB4 Protein

The ABCB4 1583_{—}1584G genotype results in a frameshift mutation that produces four premature stop codons in the translated sequence. The resultant truncated protein, shown as SEQ ID NO: 4, is approximately 50% of the wildtype ABCB4 protein (see FIG. 3). Thus, any method of detecting the truncated protein in a canine subject will indirectly detect the ABCB4 1583_{—}1584G genotype and by extension hepatobiliary disease. In such embodiments, ABCB4 translation can be measured quantitatively (e.g. by quantitative Western blot) or qualitatively (immunofluorescent or immunohistochemical labeling of ABCB4 protein in a section of hepatic tissue). Therefore, antibodies specifically reactive with the ABCB4 truncation protein set forth as SEQ ID NO: 4 can be used to detect the ABCB4 1583_{—}1584G genotype. Such evaluations can be performed, for example, in lysates prepared from cells, in fresh or frozen cells, in cells that have been smeared or touched on glass slides and then either fixed and/or dried, or in cells that have been fixed, embedded (e.g., in paraffin), and then prepared as histological sections on glass slides.

The availability of antibodies specific to the ABCB4 truncated protein will facilitate the detection and quantitation of cellular ABCB4 truncated protein by one of a number of immunoassay methods which are well known in the art and are presented in Harlow and Lane (Antibodies, A Laboratory Manual, CSHL, New York, 1988). Any standard immunoassay format (e.g., ELISA, western blot, or RIA assay) can be used to measure ABCB4 truncated polypeptide or protein levels, and to compare these with full-length (wildtype) ABCB4 expression levels in control or reference cell populations.

By way of example, an ELISA is one type of immunoassay that can be used to determine the concentration of ABCB4 truncated protein in a sample from subject. A typical ELISA format involves a specific immobilized capture antibody, sample, a labeled detection antibody, chromogens, and stop solution. Antigen will bind to the immobilized capture antibody and thus can be detected with one or more antibodies. The antibody detection technique used with an ELISA may be direct or indirect. For direct antibody visualization of the ABCB4 truncated protein, anti-truncated-ABCB4 antibody is attached to a substrate, the substrate is incubated with a sample, and the substrate is then incubated with another anti-truncated ABCB4 antibody that has been enzyme-conjugated, for example an anti-truncated-ABCB4 antibody conjugated to alkaline phosphatase or horseradish peroxidase. For indirect antibody visualization of the truncated ABCB4 protein, anti-truncated ABCB4 antibody is attached to the substrate, and the substrate is incubated with a sample. The substrate is then incubated with an unconjugated truncated ABCB4-specific antibody (primary antibody), then with an enzyme-conjugated antibody (secondary antibody) that recognizes the primary antibody. Secondary antibodies for the indirect detection of primary antibodies are often conjugated with horseradish peroxidase or alkaline phosphatase. A substrate solution is then added, acted upon by the enzyme, and effects a color change. The intensity of the color change is proportional to the amount of antigen in the original sample. Primary and secondary antibodies also can be coupled to radioactive or fluorescent tags. The intensity of radioactive or fluorescent labeling is proportional to the amount of antigen present in the original sample.

In an alternative embodiment, truncated ABCB4 can be assayed in a sample by a competition immunoassay, such as a radioimmunoassay (RIA) utilizing truncated ABCB4 standards labeled with a detectable substance, such as radiolabel, and an unlabeled antibody that specifically binds truncated ABCB4. In this assay, the labeled truncated ABCB4 standard is mixed with the truncated ABCB4-reactive antibody. Then, the sample is combined with the antibody-bound labeled truncated ABCB4 standards. The amount of unbound, labeled truncated ABCB4 is then determined. The amount of truncated ABCB4 in the sample is proportional to the amount of unbound, labeled truncated ABCB4.

Immunohistochemical or immunofluorescence techniques may also be utilized for truncated ABCB4 polypeptide or protein detection. For example, a tissue sample may be obtained from a subject, and a section stained for the presence of truncated ABCB4 using a truncated ABCB4-specific binding agent (e.g., anti-truncated ABCB4 antibody) and any standard detection system (e.g., one which includes a secondary antibody conjugated to horseradish peroxidase or fluorescent label). General guidance regarding such techniques can be found in, e.g., Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating a truncated ABCB4 protein, a biological sample of the subject, which sample includes cellular proteins, is required. Such a biological sample may be obtained from body cells, such as those present in a tissue biopsy, surgical specimens, or autopsy material.

### VI. Methods of Decreasing Gallbladder Mucocele Frequency in a Dog Population by Selection of Dogs Lacking the ABCB4 1583_{—}1584G Genotype

Also disclosed herein are methods of breeding dogs (and corresponding dog breeding programs) that utilize the described hepatobiliary disease diagnostic methods to decrease the incidence of gallbladder mucocele in a dog population. Although gallbladder mucocele is a familial disease, its symptoms are adult onset, and generally not until late into adulthood. Thus, by the time a dog is determined to have or be predisposed to gallbladder mucocele, it may have already been used for breeding purposes and therefore it may have already transmitted to the next generation an ABCB4 allele comprising the herein-described the ABCB4 1583_{—}1584G genotype.

To prevent such transmission, and thereby decrease the frequency of gallbladder mucocele in a dog population, those dogs that a breeder is considering for breeding purposes are screened for gallbladder mucocele by detecting the ABCB4 1583_{—}1584G genotype (directly or indirectly, for instance by measuring the level truncated ABCB4 protein) using at least one of the diagnostic methods described herein. In some embodiments the dog breeder has the dogs screened for the presence of the ABCB4 1583_{—}1584G genotype. In other embodiments, the dog breeder will have the dogs screened for the level of truncated ABCB4 protein expression. Those dogs that are identified as having (or susceptible to, or carrying the genetic defect for) gallbladder mucocele can then be removed from the breeding population. In this way, the ABCB4 1583_{—}1584G genotype is not transmitted and the frequency of gallbladder mucocele is reduced in future generations.

### VII. Treatment Programs for Gallbladder Mucocele-Predisposed or Affected Dogs

Currently, no medical treatment options exist for managing dogs with gallbladder mucocele, primarily because prior to this disclosure, information regarding the etiology of the disease has been lacking.

In light of the current disclosure, it will be appreciated that treatment programs can now be developed for dogs with the ABCB4 1583_{—}1584G mutation. In particular examples, subject dogs are screened for the ABCB4 1583_{—}1584G genotype by any of the methods described herein. A dog that is accordingly identified as having the ABCB4 1583_{—}1584G genotype can then be treated by dietary supplementation with a more hydrophilic and less cytotoxic bile acid than canine bile acid. Such more hydrophilic bile acids include ursodeoxycholate, alpha, beta or gamma muricholic acid and the like and can be administered by any method known to the art, including as a pharmaceutical preparation or incorporated into dog food. In particular examples the more hydrophilic bile acid is a synthetic derivative of hydrophilic bile acid, for example UPF-680, a fluorinated analog of hyodeoxycholic acid.

It will also be appreciated that the identification herein of the association between the ABCB4 1583_{—}1584G genotype and gallbladder mucocele will also allow for identification of predisposed dogs at an earlier age than previously possible. Thus, potentially affected dogs can be identified years before development of gallbladder mucocele symptoms. Such dogs can be as young as 1, 2, 3, 4, 5, 6, 7, 8 or even 9 years of age. It will be appreciated that identification of such dogs combined with the dietary supplement with less hydrophobic bile acids described herein will allow for the inhibition or possible prevention of gallbladder mucocele in predisposed dogs. In other examples, wherein the dogs have already developed gallbladder mucocele, the dietary supplementation described herein is used to inhibit the progression of the disease and decrease resultant fatalities.

### VII. Kits

This disclosure also provides kits that enable a user to diagnose gallbladder mucocele in a dog, including reagents necessary to either detect the presence of the ABCB4 1583_{—}1584G genotype or to measure expression of truncated ABCB4 protein.

Certain kits can include reagents necessary for DNA sequencing, including, but not limited to primers, DNA polymerase, dNTPs, and ddNTPs.

Other kits can include Taq polymerase and reagents necessary for allele-specific PCR, including but not limited to, amplification primers, a label for detection of the amplified template (such as a fluorescent label), nucleotides, and buffers necessary to carry out allele-specific PCR.

Other kits can include materials necessary for quantitative or qualitative detection of truncated ABCB4 protein, including antibodies that specifically recognize truncated ABCB4 protein, labeled secondary antibodies that recognize the truncated ABCB4-specific antibody, and reagents for use in detection of the label on the secondary antibody.

The materials provided in such kits may be provided in any form practicable, such as suspended in an aqueous solution or as a freeze-dried or lyophilized powder, for instance. Kits according to this invention can also include instructions, usually written instructions, to assist the user in carrying out the detection and quantification methods disclosed herein. Such instructions can optionally be provided on a computer readable medium or as a link to an internet page.

The container(s) in which the reagents are supplied can be any conventional container that is capable of holding the supplied form, for instance, microfuge tubes, ampoules, or bottles. In some applications, the reagent mixtures may be provided in pre-measured single use amounts in individual, typically disposable, tubes, microtiter plates, or equivalent containers. The containers may also be compatible with a specific automated liquid handling apparatus.

### VIII. Animal Models

Severe ABCB4 dysfunction in humans is often fatal without liver transplant at an early age. It will be appreciated that the association between dogs and the single nucleotide insertion in exon 12 of ABCB4 described herein allows dogs possessing the ABCB4 1583_{—}1584G genotype to serve as a useful model for studying the effects of treatments for severe hepatobiliary disease in humans with ABCB4 dysfunction.

Use of the dogs possessing a ABCB4 1583_{—}1584G genotype avoids confounding effects attributable to producing ABCB4 mutations through genetic engineering, and is expected to result in better acceptance as a research model by both the research community and society. It is expected that homozygous dogs will have more severe hepatobiliary disease and thus be particularly useful as animal models

Using methods described herein, individual animals are identified as being heterozygous or homozygous the ABCB4 1583_{—}1584G genotype, and these animals are used as subjects for animal model studies. In specific examples, a drug of interest (or drug candidate or other compound or mixture thereof) is administered to dogs possessing the ABCB4 1583_{—}1584G genotype, and the hepatobiliary effects are monitored systemically, for instance, the concentration of PC in bile can be monitored or the severity of gallbladder mucoceles; such monitoring can be by any method known to the art. Routes of administration include but are not limited to oral and parenteral routes, such as intravenous (iv), intraperitoneal (ip), rectal, topical, ophthalmic, nasal, and transdermal.

Effective doses of the compound(s) of interest can be determined by one of ordinary skill in the art, and may be tailored to the specific experiments being run. In some embodiments, the compound is administered with a goal of achieving tissue concentrations that are at least as high as the IC₅₀ of the compounds(s) tested. An example of such a dosage range is 0.1 to 200 mg/kg body weight. The specific dose level and frequency of dosage for any particular subject may be varied and will depend upon a variety of factors, including the activity of the specific compound, the metabolic stability and length of action of that compound, the age, body weight, mode and time of administration, and the rate of excretion of the compound.

The following examples are provided to illustrate certain particular features and/or embodiments. These examples should not be construed to limit the invention to the particular features or embodiments described.

### EXAMPLES

### Example 1

### Identification of an Association Between a Mutation in Exon 12 of ABCB4 and Canine Hepatobiliary Disease

This example shows the identification of a single base insertion mutation in the ABCB4 gene of dogs affected by gallbladder mucocele. Aspects of this example have been published in Mealey et al., Comp. Hep., 9:6, 2010, which is incorporated by reference herein in its entirety.

### Methods

Collection of DNA from affected and unaffected individuals. Collection of DNA from affected Shetland Sheepdogs was accomplished by soliciting owners' cooperation. Owners of dogs with confirmed or suspected gallbladder disease (based on a serum biochemical panel including ALT, Alkaline Phosphatase, GGT, bilirubin, triglycerides and cholesterol) were asked to submit a cheek swab, copy of the dog's pedigree, and copy of the dog's medical record. For collection of unaffected Shetland Sheepdogs, an additional request for DNA from healthy Shetland Sheepdogs (with confirmatory medical records) was made. For collection of DNA from affected dogs of any breed, records from the Washington Animal Disease Diagnostic Laboratory were searched for canine patients with histopathologic confirmation of gallbladder mucocele. For collection of DNA from unaffected dogs of any breed, a specific solicitation was made for healthy dogs (no history of gallbladder disease) over 9 years of age. A dog was considered ‘affected’ if a gallbladder mucocele was diagnosed using previously established criteria (Aguirre et al., J. Am. Vet. Med. Assoc., 231: 79-88, 2007), which included at least one of the following (in order of increasing stringency); ultrasound report by a boarded veterinary radiologist, surgical report, or histopathologic report. Dogs with abnormal serum biochemistry values indicative of gallbladder disease, but that had not undergone ultrasound, surgery, or biopsy, were considered to be neither ‘affected’ nor ‘unaffected’, and were not included in statistical calculations. Dogs with no evidence of gallbladder disease were considered ‘unaffected’.

Sequencing of Canine ABCB4. Exons 1 through 26 of canine ABCB4 (NC_{—}006596.2; GI:74031036) were sequenced after PCR amplification of genomic DNA from affected and unaffected Shetland Sheepdogs. Table 1 presents the sequences of the oligonucleotide primers used to amplify and sequence each exon. Purified PCR amplicons were sequenced with an Applied Biosystems ABI 3730 sequencer. Affected and unaffected dogs of other breeds (non-Shetland Sheepdogs) were sequenced only at exon 12.

TABLE 1
Primers used to amplify and sequence canine
ABCB4

| | | | Prod- |
|---|---|---|---|
| | | | uct |
| Exon | Forward Primer | Reverse Primer | length |
| 1 | TTCAGTTGGCTATGAAACA | AGA CTA TCT TAA AGC | 165 |
| | TTTGG | ACT GAC TCC | |
| | (SEQ ID NO: 8) | (SEQ ID NO: 9) | |
| 2 | CCA AAA AAC ATA TAG | GTC ATC TAG AAG TGC | 302 |
| | TTT TGG GGA | AAA CCA TTA AAC | |
| | (SEQ ID NO: 10) | (SEQ ID NO: 11) | |
| 3 | CCT AGT AAC ACC TAT | CTC TGT AAG TTT GCA | 202 |
| | TAA TAG TTC AGC C | ATT ATT CTC | |
| | (SEQ ID NO: 12) | (SEQ ID NO: 13) | |
| 4 | CTT CCT GAA AGA GAT | CAA AAG TAT GAC ATA | 225 |
| | GAA TAA AGA AC | AAT GAT ACA CTT AC | |
| | (SEQ ID NO: 14) | (SEQ ID NO: 15) | |
| 5 | GAA GAC CTC CTG CCT | CAC ATG TGA AAA TGT | 201 |
| | GTA ACC ACT | TCC CGT TTC | |
| | (SEQ ID NO: 16) | (SEQ ID NO: 17) | |
| 6 | CAT GAA TGT TTC TTC | GGT TCT TTG AAC CAG | 143 |
| | TCT GTC CAG | TGG AC | |
| | (SEQ ID NO: 18) | (SEQ ID NO: 19) | |
| 7 | GGC TAT GAT TAT GGA | GGT TTC TTC ACG AAT | 208 |
| | CTG TTT TCT TG | ATT AGA AAG AC | |
| | (SEQ ID NO: 20) | (SEQ ID NO: 21) | |
| 8 | GCT TAT AAC TTC TTC | GTG CAA GCC TCA AGG | 143 |
| | TTG TGT TCT TTT G | AAT TTT TTT TG | |
| | (SEQ ID NO: 22) | (SEQ ID NO: 23) | |
| 9 | CCT TAA AAG TGC AGT | GAA ATA AAA CCT GCC | 249 |
| | TGG TTG | ACA GG | |
| | (SEQ ID NO: 24) | (SEQ ID NO: 25) | |
| 10 | CGT GAA GAG TGT TCT | GCA GGG CTA ATT GGT | 177 |
| | CTT TCT CTC | AGC | |
| | (SEQ ID NO: 26) | (SEQ ID NO: 27) | |
| 11 | CTT GAT GCT TTA GAT | CTC ACT TGC CTG AAG | 278 |
| | GTC AGA TGG | TCA AAG | |
| | (SEQ ID NO: 28) | (SEQ ID NO: 29) | |
| 12 | GAG ATA CAT CAG GAG | CAG GTG TTT CGG GTT | 189 |
| | CTC CTC C | GAC TG | |
| | (SEQ ID NO: 30) | (SEQ ID NO: 31) | |
| 13 | GTA ACC CTG TTG CAT | CTC AGC ATG GCA TTA | 239 |
| | CAC AC | GCT GC | |
| | (SEQ ID NO: 32) | (SEQ ID NO: 33) | |
| 14 | CAA CTT AAC ATT TTC | GGA ATC ACT TGT GCC | 256 |
| | TCT TCT TTC AG | TGC | |
| | (SEQ ID NO: 34) | (SEQ ID NO: 35) | |
| 15 | CCA CTT TCT CCT GAT | GGT GAA GCT GGC ATG | 219 |
| | TCT CCT G | AGA AC | |
| | (SEQ ID NO: 36) | (SEQ ID NO: 37) | |
| 16 | CTC TCT CTG GCT CTC | CTC TAA TAG AAT GTG | 188 |
| | ATG | GAC TCG AG | |
| | (SEQ ID NO: 38) | (SEQ ID NO: 39) | |
| 17 | CTG ATG ATC AAA AGG | GGA CTT CTC AAG TGC | 118 |
| | GAC AAT C | ACA C | |
| | (SEQ ID NO: 40) | (SEQ ID NO: 41) | |
| 18 | GAA GGT GTG TTT TGT | CCC TTT CTG TCT CTC | 141 |
| | GCC ACA G | AAA TGG G | |
| | (SEQ ID NO: 42) | (SEQ ID NO: 43) | |
| 19 | CAT GGC TCC CTC TTT | CTC ACT GAA GCC TTC | 212 |
| | GCT TTT GC | TTT GAC CCA C | |
| | (SEQ ID NO: 44) | (SEQ ID NO: 45) | |
| 20 | CGT TAT CCA GAA GTA | CCT CAG GAA AGT ACT | 159 |
| | AAA GCC C | AGG GTC | |
| | (SEQ ID NO: 46) | (SEQ ID NO: 47) | |
| 21 | CCA GTC AAC TAC ACT | GAA CAA GTG AGT TTT | 260 |
| | AGA AGC TG | TTC CAC CC | |
| | (SEQ ID NO: 48) | (SEQ ID NO: 49) | |
| 22 | GGT AAG CAC TAT GTC | CAT TCA CCA GAC AGC | 222 |
| | TTT GGA C | AGA GAA C | |
| | (SEQ ID NO: 50) | (SEQ ID NO: 51) | |
| 23 | CAG ACC AAT TAT AAT | GCC TTA AAT AAG GTA | 227 |
| | AGC AAC ATT AAC | CTA ACT TAA GC | |
| | (SEQ ID NO: 52) | (SEQ ID NO: 53) | |
| 24 | GAT ACC CAC ATG TCA | TCC TGG TGC CAC TAC | 402 |
| | CAA TGT TCC | ATA GAC | |
| | (SEQ ID NO: 54) | (SEQ ID NO: 55) | |
| 25 | GTC CTA TAC CAA GTC | GGA AAC AGA GTG GAA | 179 |
| | ATG AGG AC | AGA CC | |
| | (SEQ ID NO: 56) | (SEQ ID NO: 57) | |
| 26 | GGA ACT AAC TGT AGA | GCT ATC TTA TCA ACA | 393 |
| | CTA TAA TGC | CCA AAT GG | |
| | (SEQ ID NO: 58) | (SEQ ID NO: 59) | |

Allele Specific PCR. To confirm the insertion mutation in exon 12 (ABCB4 1583_{—}1584G) in affected dogs, allele specific primers were designed (mutant: forward 5′-CCT GGT TCG CAA CCC TAA GAT CCG (SEQ ID NO: 5), reverse 5′-GCA ATG TGG CCT GAC AGA AAG GGG AAA TC (SEQ ID NO: 6); wild type: forward 5′-CCT GGT TCG CAA CCC TAA GAT CC (SEQ ID NO: 7), reverse 5′-GCA ATG TGG CCT GAC AGA AAG GGG AAA TC (SEQ ID NO: 6)) to amplify a 202 bp amplicon. This also allowed confirmation of individual genotype.

Statistics. Association of genotype and gallbladder mucocele status was analyzed using the frequency procedure of SAS 9.2 (SAS Institute, Cary, N.C.), specifying Fisher's exact test and exact confidence intervals for the odds ratio.

### Results

Collection of DNA of affected and unaffected individuals. To identify a possible association between a defect in the ABCB4 gene and gallbladder mucocele. DNA samples from 15 gallbladder mucocele-affected and 19 unaffected Shetland Sheepdogs were obtained for sequencing. Diagnosis of gallbladder mucocele was confirmed by ultrasound in three dogs, by surgery in five dogs, and by histopathology in seven dogs. Median age of Shetland Sheepdogs with a diagnosis of gallbladder mucocele was nine years (range 5-12), which is similar to previous reports (Aguirre et al., J. Am. Vet. Med. Assoc., 231: 79-88, 2007 and Pike et al., J. Am. Vet. Med. Assoc., 224: 1615-1622, 2004). Four dogs had blood chemistry values consistent with biliary disease but had not undergone the specific diagnostic procedures required for inclusion into the affected group (median age 4.5 years; range 2-6). Ages for the 19 unaffected Shetland Sheepdogs were not all available, but the median age for those dogs whose ages were known was nine years of age. Ages and breeds of the three affected non-Shetland Sheepdogs are as follows: Cairn Terrier (11 years), Cocker Spaniel (13 years) and Pomeranian (11 years).

Sequencing of Canine ABCB4. Sequencing of all exons (1-26) of canine ABCB4 was performed on genomic DNA from cheek swab samples or from archived liver tissue (affected dogs that were not Shetland Sheepdogs). A single base pair insertion (G) was identified in exon 12 (FIG. 1) in 14 of 15 affected Shetland Sheepdogs, one of 18 unaffected Shetland Sheepdogs, and three affected dogs of other breeds (Cairn Terrier, Cocker Spaniel, and Pomeranian). The insertion mutation (ABCB4 1583_{—}1584G) is significantly associated (P=1.54E-7) with the diagnosis of gallbladder mucocele in Shetland Sheepdogs, with an odds ratio of 252 (95% CI 11-111,180). The frame shift generated by the insertion results in four premature stop codons within exon 12. Essential structural elements of ABCB4 affected include both ATP binding sites and a putative substrate binding site (FIG. 3).

A missense mutation in exon 15 of canine ABCB4 was identified in the one affected Shetland Sheepdog that did not harbor ABCB4 1583_{—}1584G. This SNP results in a nonhomologous amino acid substitution (alanine to serine) which may affect tertiary protein structure, but the association between this polymorphism and gallbladder mucocele is not statistically significant.

Confirmation of Insertion by Allele Specific PCR. To confirm the presence of ABCB4 1583_{—}1584G insertion as well as determine the genotype of each dog, allele specific primers were designed and used to amplify the region of interest in exon 12 (FIG. 2). All dogs harboring the insertion were heterozygous at the mutant allele suggesting a dominant mode of inheritance with incomplete penetrance. None of the dogs in the study were homozygous for the mutant allele. Genotype frequencies are shown in Table 2.

TABLE 2
ABCB4 Genotype Frequencies

| | Shetland Sheepdog | Shetland Sheepdog |
|---|---|---|
| | GM* affected | GM unaffected |
| ABCB4 1583_1584G | 1 | 14 |
| (wildtype) | | |
| ABCB4 1583_1584G | 18 | 1 |
| (heterozygous) | | |
| ABCB4 1583_1584G | 0 | 0 |
| (homozygous) | | |

*GM = Gallbladder mucocele

### Example 2

### Methods of Decreasing Gallbladder Mucocele Frequency in a Dog Population

This example provides a method to decrease the incidence/frequency of gallbladder mucocele in dogs through screening and selective breeding.

Although the development of gallbladder mucocele is an inherited disease, its symptoms are adult onset. Thus, symptoms in a gallbladder mucocele-predisposed dog may not appear until long after that dog has been used for breeding and has already passed on the ABCB4 1583_{—}1584G genotype to the next generation.

To decrease the frequency of gallbladder mucocele in a dog population, candidate breeding dogs are screened by any method for detecting the ABCB4 1583_{—}1584G genotype and thus detecting a predisposition for gallbladder mucocele. After isolating DNA from the candidate breeding dogs, the 1583_{—}1584G genotype can be identified by any method known to the art of detecting nucleotide insertions in a gene sequence, for example sequencing of a region of the ABCB4 gene including nucleotides 1582 and 1583 or allele-specific PCR using primers that will amplify the mutant allele but not the wildtype ABCB4 allele. Based on this screen, dogs found to have the ABCB4 1583_{—}1584G genotype are deemed unsuitable for breeding. By breeding only those dogs that are determined to not be predisposed to gallbladder mucocele (that is, dogs that have the 1583_{—}1584G genotype), the incidence of gallbladder mucocele in the particular dog population will be decreased in future generations.

### Example 3

### Methods of Inhibiting Development of Gallbladder Mucocele

In general, gallbladder mucocele do not develop in dogs until late adulthood (generally dogs nine years old or greater). The present disclosure allows for the detection of dogs that are predisposed to gallbladder mucocele many years before disease onset. This example describes treatment of dogs predisposed to developing gallbladder mucocele to inhibit or even prevent the development of the disease.

To inhibit the development of gallbladder mucocele, a dog is screened for the presence of the ABCB4 1583_{—}1584G genotype by any of the methods described above. A dog that has the ABCB4 1583_{—}1584G genotype is considered to be predisposed to developing gallbladder mucocele. Once identified as being predisposed to gallbladder mucocele, the predisposed dog is then fed a diet supplemented with bile salts that are more hydrophilic than native canine bile salts. Examples of such bile salts include ursodeoxycholate and muricholic acid. By this method the development of gallbladder mucocele in predisposed dogs is inhibited. One of skill in the art will appreciate that supplementing the diet of dogs that have already been diagnosed with having gallbladder mucocele (and have the ABCB4 1583_{—}1584G genotype) will also benefit from a diet supplemented by less hydrophobic (and as such less cytotoxic) bile salts as described above.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of detecting predisposition to or presence of hepatobiliary disease in a canine subject, comprising: detecting a nucleotide insertion in the ABCB4 gene, wherein the nucleotide insertion results in premature termination of ABCB4 translation.

2. The method of claim 1, wherein the nucleotide insertion is located within or 5′ (upstream) of exon 12 of the ABCB4 gene.

3. The method of claim 1, wherein the nucleotide insertion is located between nucleotides 1582 and 1583 of ABCB4 nucleic acid sequence as set forth as SEQ ID NO: 1.

4. The method of claim 3, wherein the canine subject is selected from the group consisting of Shetland Sheepdog, Cairn Terrier, Cocker Spaniel, and Pomeranian.

5. The method of claim 1, wherein the hepatobiliary disease comprises gallbladder mucocele.

6. The method of claim 3, wherein detecting the nucleotide insertion in the ABCB4 gene comprises:
isolating DNA from the canine subject; and
sequencing a region of the DNA including the nucleotide sequence corresponding to nucleotides 1582-1583 of SEQ ID NO: 1.

7. The method of claim 6, further comprising amplifying the region of the DNA including the nucleotide sequence corresponding to nucleotides 1582-1583 of SEQ ID NO: 1, prior to sequencing.

8. The method of claim 1, wherein detecting the nucleotide insertion in the ABCB4 gene comprises allele-specific PCR comprising:
isolating DNA from the canine subject;
contacting the DNA with a primer pair comprising a forward primer and a reverse primer under conditions sufficient to amplify ABCB4 DNA, wherein either the forward or the reverse primer comprises an oligonucleotide that specifically hybridizes to a region of the ABCB4 DNA comprising the nucleotide insertion; and
detecting amplified ABCB4 DNA comprising the nucleotide insertion.

9. The method of claim 8, wherein the forward primer comprises an oligonucleotide comprising a sequence no more than three nucleotides variant from SEQ ID NO: 5, except the G at position 24 is invariant, and wherein the reverse primer comprises an oligonucleotide comprising a sequence no more than three nucleotides variant from SEQ ID NO: 6.

10. A method of selectively breeding dogs to decrease the frequency of hepatobiliary disease in a dog population, the method comprising:
identifying dogs in a breeding population that have a predisposition to hepatobiliary disease by the method of claim 1;
selecting for breeding those dogs that do not have a predisposition to hepatobiliary disease; and
breeding only selected dogs, thereby decreasing the frequency of hepatobiliary disease in the dog population.

11. The method of claim 10, wherein the nucleotide insertion is located between nucleotides 1582 and 1583 of ABCB4 sequence as set forth as SEQ ID NO: 1.

12. The method of claim 10, wherein the canine subject is selected from the group consisting of Shetland Sheepdog, Cairn Terrier, Cocker Spaniel, and Pomeranian.

13. The method of claim 10, wherein the hepatobiliary disease comprises gallbladder mucocele.

14. The method of claim 11, wherein detecting the nucleotide insertion in the ABCB4 gene comprises:
isolating DNA from the canine subject; and
sequencing a region of the DNA including the nucleotide sequence corresponding to nucleotides 1582-1583 of SEQ ID NO: 1.

15. The method of claim 10, wherein detecting the nucleotide insertion in the ABCB4 gene comprises allele-specific PCR comprising:
isolating DNA from the canine subject;
contacting the DNA with a primer pair comprising a forward primer and a reverse primer under conditions sufficient to amplify ABCB4 DNA, wherein either the forward or the reverse primer comprises an oligonucleotide that specifically hybridizes to a region of the ABCB4 DNA comprising the nucleotide insertion; and
detecting amplified ABCB4 DNA comprising the nucleotide insertion.

16. A kit for detecting the predisposition to or presence of hepatobiliary disease in a canine subject, comprising:
at least one agent capable of detecting an ABCB4 1583_{—}1584G genotype in the canine subject comprising
(a) an oligonucleotide that specifically hybridizes to a region of ABCB4 DNA comprising an insertion between nucleotides 1582 and 1583 of SEQ ID NO: 1; or
(b) an antibody that specifically recognizes a truncated ABCB4 polypeptide set forth as SEQ ID NO: 4 and does not recognize a full-length ABCB4 polypeptide; and
instructions for using the agent to detect the ABCB4 1583_{—}1584G genotype.

17. The kit of claim 16, wherein the hepatobiliary disease comprises gallbladder mucocele.

18. The kit of claim 16, wherein the oligonucleotide comprises a sequence no more than three nucleotides variant from SEQ ID NO: 5, except the G at position 24 is invariant.

19. A treatment program to inhibit or prevent development of gallbladder mucocele in a canine subject, the treatment program comprising:
detecting a canine subject that is predisposed to or has a hepatobiliary disease comprising a gallbladder mucocele by the method of claim 5; and
administering to the canine subject bile salts that are more hydrophilic than canine bile salts;
thereby inhibiting the development of gallbladder mucocele in the canine subject.

20. The treatment program of claim 20, wherein the canine subject is no older than 1, 2, 3, 4, 5, 6 or 7 years old.
